(19) United States
(12) Patent Application Publication
(10) Pub. No.: US 2022/0284984 A1
(43) Pub. Date: Sep. 8, 2022

(54) SOMATIC VARIANT CALLING FROM AN UNMATCHED BIOLOGICAL SAMPLE

(71) Applicant: Personalis, Inc., Menlo Park, CA (US)

(72) Inventors: Patrick Jongeneel, Menlo Park, CA (US); Nicholas Phillips, Menlo Park, CA (US); Jason Harris, Menlo Park, CA (US)

(73) Assignee: Personalis, Inc., Menlo Park, CA (US)

(21) Appl. No.: 17/735,906

(22) Filed: May 3, 2022

Related U.S. Application Data
(63) Continuation of application No. PCT/US2020/058955, filed on Nov. 4, 2020.
(60) Provisional application No. 62/931,100, filed on Nov. 5, 2019.

(51) Int. Cl. G16B 20/20; G16B 40/20

(52) U.S. Cl. CPC G16B 20/20; G16B 40/20

(57) ABSTRACT

Methods for somatic variant calling from an unmatched biological samples is provided. The method can include obtaining nucleic acid sequence data corresponding to a biological sample of a subject. The method can also include aligning the nucleic acid sequence data to a reference genome. The method can also include identifying, based on the aligned nucleic acid sequence data, a set of candidate variants in said nucleic acid sequence data. The set of candidate variants may include one or more somatic variants and one or more germline variants. The method can also include, without using a nucleic acid sequencing data from a matching biological sample of the subject, processing the set of candidate variants using a trained machine-learning model to identify the somatic variants. The method can also include outputting a report that identifies the somatic variants.

### CROSS REFERENCE TO RELATED APPLICATIONS

The present application is a continuation of International Application No. PCT/US2020/058955 filed Nov. 4, 2020, which claims priority to and the benefit of U.S. Provisional Patent Application No. 62/931,100, filed on Nov. 5, 2019, which is hereby incorporated by reference herein in its entirety for all purposes.

### FIELD

This disclosure generally relates to systems and methods for identifying somatic variants in a biological sample. More specifically, but not by way of limitation, this disclosure relates to identifying somatic variants in a biological sample by using trained machine-learning models to filter false positives from a detected set of candidate variants.

### BACKGROUND OF THE INVENTION

Somatic variants in a DNA sequence can indicate one or more mutations that contribute to a development of cancer. For many analyses of tumor samples, identifying somatic variants facilitates an improvement in cancer diagnosis, prognosis, treatment decisions, and treatment efficacy. To identify somatic variants in a biological sample, germline sequence variants and somatic variants can be distinguished. Conventional somatic-variant calling techniques rely heavily on contrasting evidence for variation between a tumor sample and a matching normal sample. However, there are several instances in which the matching normal sample is unavailable for analysis.

Accordingly, there is a need for accurately identifying somatic variants in a biological sample and for distinguishing somatic variants from germline variants, without relying on a normal control sample.

### BRIEF SUMMARY OF THE INVENTION

In some embodiments, a method of identifying somatic variants from a biological sample is provided. The method can include obtaining nucleic acid sequence data corresponding to a biological sample of a subject. The method can also include aligning the nucleic acid sequence data to a reference genome (e.g., generated based on samples from other subjects). The method can also include identifying, based on the aligned nucleic acid sequence data, a set of candidate variants in said nucleic acid sequence data. In some instances, the set of candidate variants includes one or more somatic variants and one or more germline variants.

The method can also include, without using a nucleic acid sequencing data from a matching biological sample of the subject, processing the set of candidate variants using a trained machine-learning model to identify the somatic variants. The matching biological sample of the subject indicates an absence of tumor. The method can also include outputting a report that identifies the somatic variants.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE FIGURES

Features, embodiments, and advantages of the present disclosure are better understood when the following Detailed Description is read with reference to the following figures. The patent or application file contains at least one drawing executed in color. Copies of this patent or patent application publication with color drawing(s) will be provided by the Office upon request and payment of the necessary fee.

FIG. 1 shows an example interface that is configured to identify somatic variants in paired tumor/normal sequence data, in accordance with some embodiments

FIG. 2 shows a plot that identifies precision and recall difference values between a trained gradient boosted decision tree model and baseline, in accordance with some embodiments.

FIG. 3 illustrates two classification models that can be trained to identify somatic variants in an unmatched biological sample, in accordance with some embodiments.

FIG. 4 shows a precision-recall curve corresponding to a trained filtering model for filtering out false positives from a set of candidate somatic variants, in accordance with some embodiments.

FIG. 5 shows a Shapley Additive exPlanations (SHAP) plot 500 that identifies which attributes from the attribute table affected the output of a trained filtering model, in accordance with some embodiments.

FIG. 6 shows a precision-recall curve corresponding to a trained rescue model for filtering out false negatives from a set of candidate somatic variants, in accordance with some embodiments.

FIG. 7 shows a SHAP plot that identifies which attributes from the attribute table affected the output of a trained rescue model, in accordance with some embodiments.

FIG. 8 shows a comparison in the performance of a machine-learning model with a filtering model and a rescue model before and after training and threshold adjustment, in accordance with some embodiments.

FIG. 9 illustrates a flowchart for identifying somatic variants in an unmatched biological sample, in accordance with some embodiments.

FIG. 10 illustrates an example of a computer system for implementing some of the embodiments disclosed herein.

### DETAILED DESCRIPTION

### I. Overview

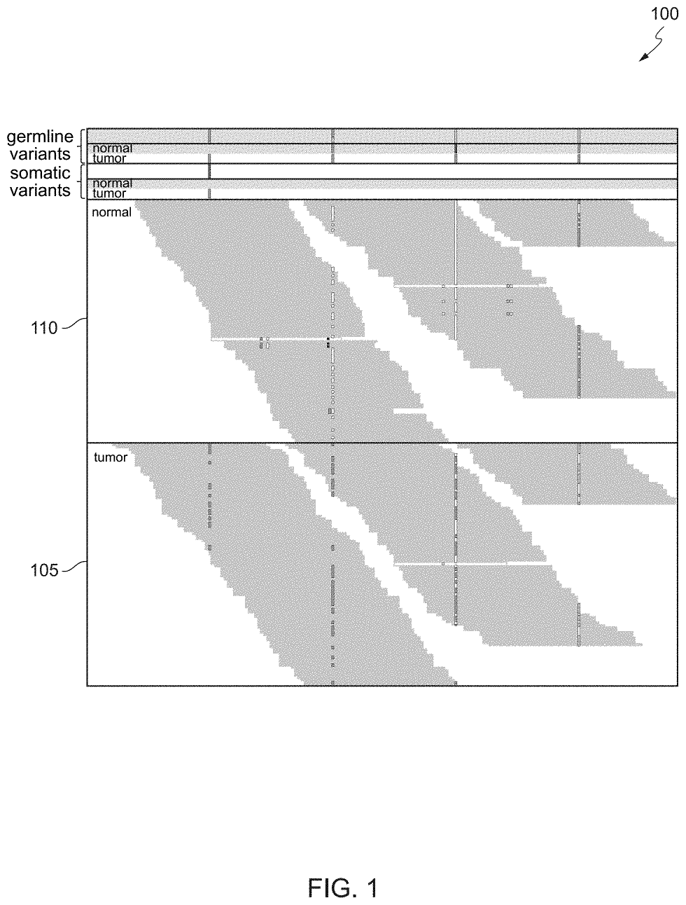

As described above, predicting somatic variants of a biological sample becomes difficult when a matching normal sample is unavailable for analysis. To illustrate, FIG. 1 shows an example interface 100 that is configured to identify somatic variants in paired tumor/normal sequence data, in accordance with some embodiments. The example interface 100 can include a bottom panel representing nucleic acid sequence data of a tumor sample 105 and a top panel representing nucleic acid sequence data of a normal sample 110. The gray bars may represent overlapping sequence reads that are aligned to a reference genome. Candidate variants can be highlighted within the reads using different colors. In the upper panel of reads, three variants can be seen that are present in 50% to 100% of reads. As these reads are from a matching normal sample, these variants can be identified as germline variants. In the lower panel of reads, the same three variants can be identified, and an additional variant is present in a subset of reads (identified by a box). As this variant is present in the tumor sample but not in the matching normal sample, it can be identified as a somatic variant.

As shown in FIG. 1, conventional somatic-variant calling techniques rely on contrasting evidence for variation between a tumor sample and a matching normal sample of a subject. An absence of the matching normal sample 110 prevents the identification of the somatic variants in the tumor sample 105, which may greatly reduce the accuracy of the conventional somatic-variant calling techniques. For example, removing the matching normal sample 100 from the example diagram 100 may cause difficulties in determining which of the candidate variants in the bottom panel are germline variants and which are somatic variants. A lack of the matching normal sample 110 may increase a quantity of false positives (e.g., germline variants) in determining the somatic variants. In some instances, false positives caused by germline contamination (for example) in the somatic variant calling output are substantially increased.

To address at least the above deficiencies of conventional systems, the present techniques can be used to identify somatic variants in an unmatched biological sample and to distinguish the somatic variants from germline variants. A trained machine-learning model that includes one or more classification models can be used to predict somatic variants based on features extracted from nucleic acid sequencing data obtained from the unmatched biological sample. In some instances, additional sources of data (e.g., databases) are used to predict the somatic variants. For example, a high-sensitivity algorithm can be used to identify candidate variants in the nucleic acid sequencing data. An attribute table can be generated, in which the attribute table may include one or more features identified for each candidate variant. The trained machine-learning model can be used to identify somatic variants based on the contents of the attribute table. A report identifying the somatic variants can be outputted. In some instances, the report includes a diagnostic report, prognostic report, and/or a treatment recommendation.

Nucleic acid sequence data of a biological sample of a subject can be obtained. In some embodiments, the sequencing data is from a tumor sample. Sequencing can include whole exome sequencing. In some embodiments, the sequencing can include whole genome sequencing. In some embodiments, the sequencing includes shotgun sequencing. In some embodiments, the sequencing includes sequencing select parts of the genome or exome.

The nucleic acid sequence data can be aligned to a reference genome. As used herein, the reference genome corresponds to nucleic acid sequence corresponding to a representative example of the set of genes in one idealized individual organism of a species. Based on the aligned nucleic acid sequence data, a set of candidate variants in the nucleic acid sequence data can be identified. In some instances, the set of candidate variants includes one or more somatic variants and one or more germline variants. As used herein, a “somatic variant” refers to an alteration in DNA that occurs after conception and is not present within the germline. The somatic variant can occur in any of the cells of the body except the germ cells (sperm and egg) and therefore cannot be inherited. In addition, a “germline variant” refers to a gene change in a reproductive cell (egg or sperm) that becomes incorporated into the DNA of every cell in the body of the offspring. A variant (or mutation) contained within the germline can be passed from parent to offspring, and is, therefore, hereditary. In some instances, the somatic variants, instead of the germline variants, indicate a presence or a level of cancer in the subject.

An attribute table (for example) can be generated, in which the attribute table can include a number of features for each candidate variant. In some embodiments, the attribute table includes attributes from sequencing data that corresponds to a particular candidate variant. The attribute table can include attributes from a file including processed sequencing data. In some embodiments, the attribute table includes one or more attributes as follows: (a) pileup attributes from a BCFtools output file; (b) allelic frequency data; (c) base quality data; (d) read depth data; (e) an estimation of tumor cellularity (which may be calculated based on a B allele frequency distribution); (f) predicted germline variants; (g) predicted somatic variants; (h) copy number alteration data; (i) population frequency data from one or more databases; (j) data from at least one database selected from the group consisting of Cosmic, GnomAD, Dbsnp, and Mills Indels; (k) data regarding the presence of candidate somatic variants in problematic regions of the genome; and (1) data regarding the presence of candidate somatic variants in homopolymers.

Without using a nucleic acid sequencing data from a matching normal sample of the subject, the set of candidate variants can be processed using a trained machine-learning model to identify the somatic variants. In some instances, the trained machine-learning model includes gradient-boosted decision trees that facilitate significant reduction of false positive rate corresponding to somatic-variant calls. Thus, the present technique can detect somatic variants from unmatched biological samples with enhanced sensitivity and specificity compared to conventional heuristic techniques. In some embodiments, the trained machine-learning model includes a two model classification method. The machine-learning model may include a filtration model that filters out false positives. The machine-learning model may include a rescue model that rescues false negatives. In some embodiments, the somatic variants are predicted with a precision of at least 0.5. In some embodiments, the somatic variants are predicted with a recall of at least 0.5. In some embodiments, the machine-learning model includes hyperparameters that are tuned by randomized search. In some embodiments, the hyperparameters include a max depth of 5-100, a minimum data in leaf of 2-50, and at least 2-2048 leaves. In some embodiments, the filtration model includes a threshold value of about 0.45. In some embodiments, the rescue model includes a threshold value of about 0.9995.

A report that identifies the somatic variants can be output. In some embodiments, the report includes information identifying at least one diagnostic marker, at least one prognostic marker. In some embodiments, an absence of a somatic variant, a treatment recommendation, a recommendation to administer a treatment to the human subject, and/or a recommendation to not administer a treatment to the human subject. In some embodiments, the recommended treatment is administered to the human subject.

Accordingly, embodiments of the present disclosure provide a technical advantage over conventional systems by increasing the accuracy of somatic variant calling from unmatched biological samples. Such techniques could potentially improve the accuracy of diagnostic, prognostic and/or treatment recommendation reports generated based on sequencing data from unmatched biological samples. Such techniques may also reduce the costs and resources required for identification of somatic variants in tumors.

While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

### II. Machine-Learning Models for Somatic Variant Calling from Unmatched Biological Samples

A. Training Machine-Learning Models to Identify Somatic Variants from Unmatched Biological Samples

The machine-learning model for identifying somatic variants from an unmatched biological sample can be trained using training dataset that includes tumor samples and normal samples that correspond to the tumor samples. For example, a training dataset may include sequencing data obtained for 350 tumor/normal sample pairs (for example). DNA from the training samples are extracted, processed, and subjected to whole exome sequencing. Sequencing reads are subjected to quality control processing (e.g., via FastQC) to provide FASTQ files. FASTQ files are aligned to a reference genome to generate a BAM files. BCFtools is used to identify a set of candidate somatic variants for each training sample at high sensitivity. The set of candidate somatic variants will include false positives, e.g., germline variants.

For the set of candidate somatic variants, an attribute table is generated that includes a plurality of features for each candidate variant (e.g., about 10-20 features). The attribute table can include: (i) pileup attributes from the initial BCFtools output, such as allelic frequency (e.g., B allele frequency), base quality, read depth, etc.; (ii) an estimate of tumor purity determined using a deep learning neural network, based on whole exome B allele frequency distribution in the sample; (iii) whether the variant is identified as a germline variant using GATK HaplotypeCaller; (iv) somatic copy number alteration (CNA) state for each variant site; (v) the frequency of the variant in populations (e.g., in healthy human populations and/or in cancer exomes from databases such as Cosmic, GnomAD, Dbsnp, Mills Indels, etc.); (vi) presence of the variant in problematic regions, such as in homopolymers; and (vii) whether the variant is identified by standard somatic callers (run in the single-tumor context), e.g., MuTect and MuTect2.

Classification labels are created based on the presence of a candidate variant in VCF files generated by MuTect or MuTect 2 using default parameters with in-house reporting criteria applied. Matching normal samples are considered by MuTect/MuTect 2 for the generation of these classification labels, which identify “true” somatic variants, and are used to evaluate model performance.

In some instances, the machine-learning models are trained and tested to identify somatic variants based on the contents of the attribute table. The training dataset can be split into training (90%) and test (10%) sets. In some embodiments, the trained machine-learning model is trained with the training dataset to achieve one or more predetermined performance levels for estimating tumor purity. The one or more predetermined performance levels include the following:
a precision of at least about 0.2, 0.25, 0.3, 0.35, 0.4, 0.45, 0.5, 0.55, 0.6, 0.65, 0.7, 0.75, 0.8, 0.85, 0.9, 0.95, or more. In some instances, the trained machine-learning model is trained to predict somatic variants with a precision of about 0.2-1.0, 0.2-0.9, 0.2-0.8, 0.2-0.7, 0.2-0.6, 0.2-0.5, 0.2-0.4, 0.2-0.3, 0.3-1.0, 0.3-0.9, 0.3-0.8, 0.3-0.7, 0.3-0.6, 0.3-0.5, 0.3-0.4, 0.4-1.0, 0.4-0.9, 0.4-0.8, 0.4-0.7, 0.4-0.6, 0.40.5, 0.5-1.0, 0.5-0.9, 0.5-0.8, 0.5-0.7, 0.5-0.6, 0.6-1.0, 0.6-0.9, 0.6-0.8, 0.6-0.7, 0.7-1.0, 0.7-0.9, 0.7-0.8, 0.8-1.0, 0.8-0.9, or 0.9-1.0;
a recall of at least about 0.2, 0.25, 0.3, 0.35, 0.4, 0.45, 0.5, 0.55, 0.6, 0.65, 0.7, 0.75, 0.8, 0.85, 0.9, 0.95, or more. In some instances, the trained machine-learning model is trained to predict somatic variants with a recall of about 0.2-1.0, 0.2-0.9, 0.2-0.8, 0.2-0.7, 0.2-0.6, 0.2-0.5, 0.2-0.4, 0.2-0.3, 0.3-1.0, 0.3-0.9, 0.3-0.8, 0.3-0.7, 0.3-0.6, 0.3-0.5, 0.3-0.4, 0.4-1.0, 0.4-0.9, 0.4-0.8, 0.4-0.7, 0.4-0.6, 0.4-0.5, 0.51.0, 0.5-0.9, 0.5-0.8, 0.5-0.7, 0.5-0.6, 0.6-1.0, 0.6-0.9, 0.6-0.8, 0.6-0.7, 0.7-1.0, 0.7-0.9, 0.7-0.8, 0.8-1.0, 0.8-0.9, or 0.9-1.0;
an F1 score (e.g., a macro averaged F1 classification score) of at least about 0.2, 0.25, 0.3, 0.35, 0.4, 0.45, 0.5, 0.55, 0.6, 0.65, 0.7, 0.75, 0.8, 0.85, 0.86, 0.87, 0.88, 0.89, 0.9, 0.91, 0.92, 0.93, 0.94, 0.95, 0.96, 0.97, 0.98, 0.99, 0.995, or more. In some instances, the trained machine-learning model is trained to predict somatic variants with an F1 score of about 0.2-1.0, 0.2-0.99, 0.2-0.95, 0.20.9, 0.2-0.8, 0.2-0.7, 0.2-0.6, 0.2-0.5, 0.2-0.4, 0.2-0.3, 0.3-1.0, 0.3-0.99, 0.3-0.95, 0.3-0.9, 0.3-0.8, 0.3-0.7, 0.3-0.6, 0.3-0.5, 0.3-0.4, 0.4-1.0, 0.4-0.99, 0.4-0.95, 0.4-0.9, 0.4-0.8, 0.4-0.7, 0.4-0.6, 0.4-0.5, 0.5-1.0, 0.5-0.99, 0.5-0.95, 0.5-0.9, 0.5-0.8, 0.5-0.7, 0.5-0.6, 0.6-1.0, 0.6-0.99, 0.6-0.95, 0.6-0.9, 0.6-0.8, 0.6-0.7, 0.7-1.0, 0.7-0.99, 0.7-0.98, 0.7-0.97, 0.7-0.96, 0.7-0.95, 0.7-0.9, 0.7-0.8, 0.8-1.0, 0.8-0.99, 0.8-0.98, 0.8-0.97, 0.8-0.96, 0.8-0.95, 0.8-0.9, 0.9-1.0, 0.9-0.99, 0.9-0.98, 0.90.97, 0.9-0.96, or 0.9-0.95;
a false positive rate of at most about 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, or 50%; and
an area under the curve-receiver operating characteristics (AUC-ROC) of at least about 0.1, 0.2, 0.3, 0.4, 0.45, 0.5, 0.55, 0.6, 0.7, 0.8, 0.9, 0.95, 0.96, 0.97, 0.98, 0.99, 0.995, 0.999, 0.9995, 0.9999, or more. In some cases, the trained machine-learning model is trained to achieve an AUC-ROC of at most about 0.8, 0.9, 0.95, 0.99, 0.995, 0.999, 0.9995, 0.9999, or less. In some cases, the trained model is trained to achieve an AUC-ROC of about 0.5-1.0, 0.5-0.9995, 0.5-0.999. 0.5-0.99, 0.5-0.95, 0.5-0.9, 0.50.8, 0.5-0.7, 0.5-0.6, 0.6-1.0, 0.6-0.9995, 0.6-0.99, 0.6-0.95, 0.6-0.9, 0.6-0.8, 0.6-0.7, 0.7-1.0, 0.7-0.9999, 0.7-0.9995, 0.7-0.999, 0.7-0.99, 0.7-0.98, 0.7-0.97, 0.7-0.96, 0.7-0.95, 0.7-0.9, 0.7-0.8, 0.8-1.0, 0.8-0.9999, 0.8-0.9995, 0.8-0.999, 0.8-0.99, 0.8-0.98, 0.8-0.97, 0.8-0.96, 0.8-0.95, 0.8-0.9, 0.9-1.0, 0.9-0.9999, 0.9-0.9995, 0.9-0.999, 0.9-0.99, 0.9-0.98, 0.9-0.97, 0.9-0.96, or 0.90.95. In some cases, the trained model is trained to achieve an AUC-ROC of about 0.5, 0.6, 0.7, 0.8, 0.85, 0.9, 0.95, 0.96, 0.97, 0.98, 0.99, 0.995, 0.997, 0.999, 0.9995, or 0.9999. In some embodiments, a high AUC-ROC value indicates a higher likelihood of discriminating true positive variants from true negative variants.

The trained machine-learning model can use one or more threshold values. Threshold values for a model can be selected based on (for example) maximizing mean sample AUC of the precision recall curve. In some cases, a filtering model uses a threshold value of at least about 0.1, 0.2, 0.3, 0.4, 0.45, 0.5, 0.55, 0.6, 0.7, 0.8, 0.9, 0.95, or 0.99, or more.

B. Training a Machine-Learning Model Framework with One Classification Model for Identifying Somatic Variants

The trained machine-learning model can correspond to one or more classification models. For example, the trained machine-learning model can correspond to 1, 2, 3, 4, 5, 6, 7, 8, 9, or 10 models. In some embodiments, one classification model is trained and tested to identify somatic variants from the attribute table. The classification model can include a gradient-boosted decision tree, which may be trained to predict somatic variants using an XGBoost framework (for example). The model's hyperparameters can be tuned in order to maximize macro averaged F1 classification score.

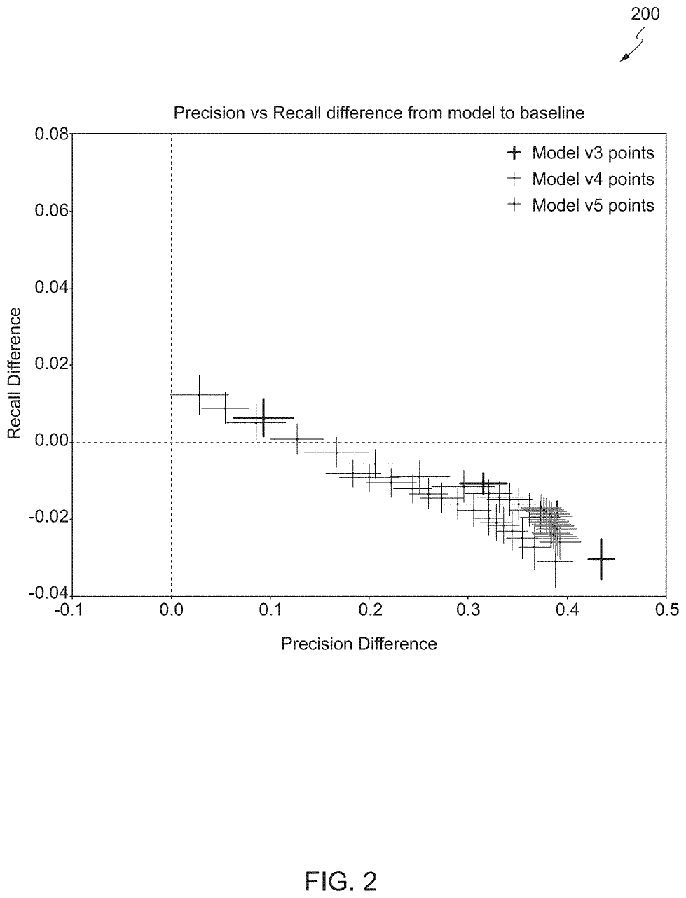

FIG. 2 shows a plot 200 that identifies precision and recall difference values between a gradient boosted decision tree model and baseline, in accordance with some embodiments. After training, the trained machine-learning model can demonstrate an increased average F1 score compared to baseline. The trained machine-learning model can achieve a high AUC-ROC (area under the curve-receiver operating characteristics) of 0.997, indicating an ability discriminate true positive variants from true negative variants. The results in FIG. 2 demonstrate the feasibility of predicting somatic variants from unmatched tumor sequencing data using the trained machine-learning model, and indicate that increased accuracy may be achieved with a model that allows for increased control of thresholding.

C. Training a Machine-Learning Model Framework with Two Classification Models for Identifying Somatic Variants

In some embodiments, the trained machine-learning model corresponds to two classification models, each of which is trained and tested to identify somatic variants from the attribute table. For increased control of thresholding, the somatic variant classification problem is decomposed into two sub-problems: (1) filter out false positives in tumor-only calls from each variant caller, and (2) rescue false negative candidate variants not present in tumor-only calls.

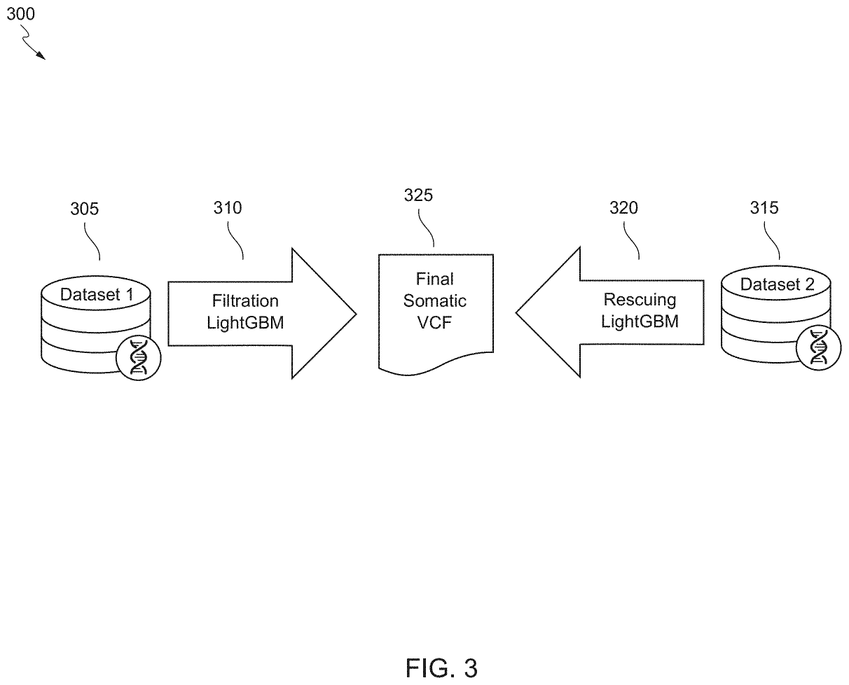

FIG. 3 illustrates two classification models 300 that can be trained to identify somatic variants in an unmatched biological sample, in accordance with some embodiments. For increased control of thresholding, the somatic variant classification problem can be decomposed into two sub-problems: (1) filter out false positives in tumor-only calls from each variant caller; and (2) rescue false negative candidate variants not present in tumor-only calls. The attribute table can thus be divided into two training datasets. In some instances, the two models are trained using a gradient boosting framework (e.g., a LightGBM framework).

A first training dataset 305 may include candidate variants that are identified by another variant-detection algorithm in tumor-only context (e.g., MuTect, MuTect2). A filtering model 310 can be trained to filter false positives out of the first training dataset. In some instances, the first training dataset 305 includes a majority of the training data set, e.g., approximately 71% of tumor-normal calls.

A second training dataset 315 may include a remainder of the candidate variants. A rescue model 320 can be trained to rescue false negatives from the second training dataset. In some instances, the rescue model 320 is trained to distinguish false negatives and true negatives, in which the false negatives correspond to those variant-detection algorithms failed to identify.

In some instances, the two classification models are trained using a gradient boosting framework (e.g., a LightGBM framework). Classification results from both of these classification models can be combined to produce a final set of somatic variants 325. The final set of somatic variants may then be used to train the classification models 310 and 320. In some instances, training the classification models 310 and 320 includes tuning one or more hyperparameters (e.g., a learning rate). During training, 300 iterations of a randomized search are used over the following set of hyperparameters for a given classification problem: (i) max depth: 5-100; (ii) minimum data in leaf: 3-50; and (iii) number of leaves: 3-2048 (log scale). Each iteration can train each of the classification models, which can be followed by a stratified 5-fold cross validation. Model averaging on the five best-fit cross validation models according to AUC-ROC (area under the curve-receiver operating characteristics) can then be applied to the test dataset.

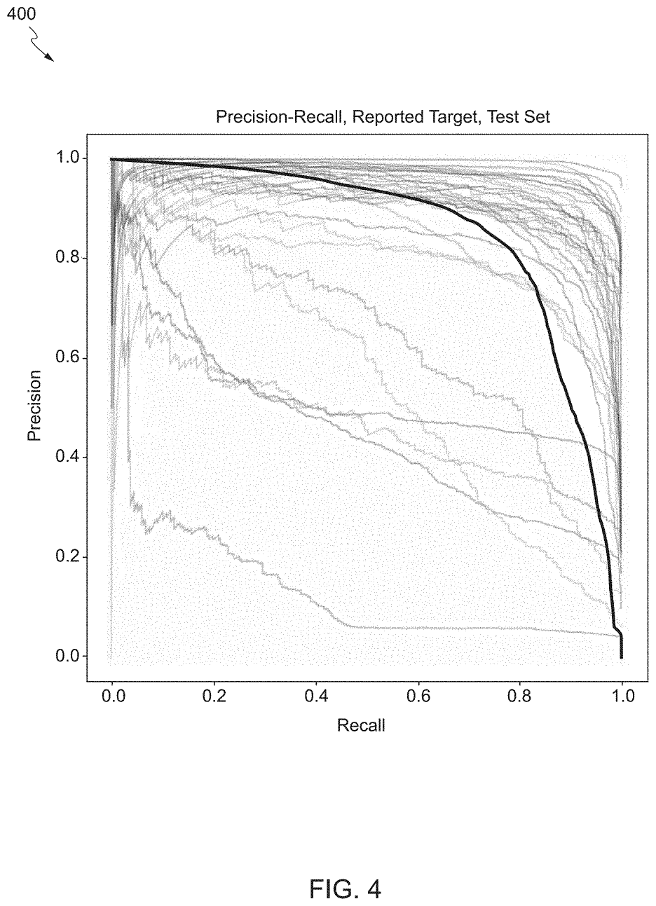

FIG. 4 shows a precision-recall curve 400 corresponding to a trained filtering model for filtering out false positives from a set of candidate somatic variants, in accordance with some embodiments. As shown in FIG. 4, the precision-recall curve 400 shows the ability of the filtering model to filter out most of the false positives from dataset. Noise in the precision-recall curve was observed due to fluctuating positive class support, while AUC-ROC remains fairly constant.

Threshold values for the filtering model 310 can be selected based on maximizing mean sample AUC of the precision recall curve. For example, a threshold value of 0.45 can be selected for the filtering model 310. In some cases, the filtering model 310 includes a threshold value of at most about 0.3, 0.4, 0.45, 0.5, 0.55, 0.6, 0.7, 0.8, 0.9, 0.95, or 0.99, or less. In some cases, the filtering model 310 includes a threshold value of about 0.2-1.0, 0.2-0.99, 0.2-0.95, 0.2-0.9, 0.2-0.8, 0.2-0.7, 0.2-0.6, 0.2-0.5, 0.2-0.4, 0.2-0.3, 0.3-1.0, 0.3-0.99, 0.3-0.95, 0.3-0.9, 0.3-0.8, 0.3-0.7, 0.3-0.6, 0.3-0.5, 0.3-0.4, 0.4-1.0, 0.4-0.99, 0.4-0.95, 0.4-0.9, 0.4-0.8, 0.4-0.7, 0.4-0.6, 0.4-0.5, 0.5-1.0, 0.5-0.99, 0.5-0.95, 0.5-0.9, 0.5-0.8, 0.5-0.7, 0.5-0.6, 0.6-1.0, 0.6-0.99, 0.6-0.95, 0.6-0.9, 0.6-0.8, 0.6-0.7, 0.7-1.0, 0.7-0.99, 0.7-0.98, 0.7-0.97, 0.7-0.96, 0.7-0.95, 0.7-0.9, 0.7-0.8, 0.8-1.0, 0.8-0.99, 0.8-0.98, 0.8-0.97, 0.8-0.96, 0.8-0.95, 0.8-0.9, 0.9-1.0, 0.9-0.99, 0.9-0.98, 0.9-0.97, 0.9-0.96, or 0.9-0.95. In some cases, the filtering model 310 includes a threshold value of about 0.1, 0.2, 0.3, 0.4, 0.45, 0.5, 0.55, 0.6, 0.7, 0.8, 0.9, 0.95, or 0.99. In some embodiments, the filtering model 310 includes a threshold value of about 0.4 to about 0.5. In some embodiments, the filtering model 310 includes a threshold value of about 0.45.

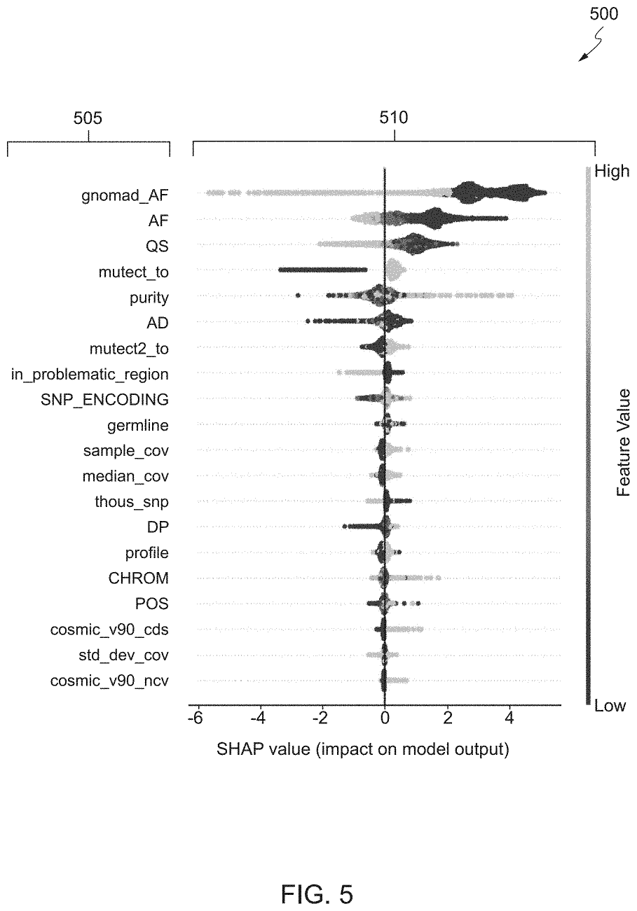

FIG. 5 shows a Shapley Additive exPlanations (SHAP) plot 500 that identifies which attributes from the attribute table affected the output of a trained filtering model, in accordance with some embodiments. The SHAP plot 500 depicts graphical information that identifies an extent to which each attribute in the attribution table contributed to the identification of false positives of somatic variants in the biological sample. The SHAP plot 500 includes a left portion 505 that identifies a plurality of features derived from the attribute table, in which each row corresponds to one of a plurality of attributes determined for a given candidate variant. The SHAP plot 500 also includes a right portion 510 that identifies, for a given attribute, an extent of contribution to the identification of the false positives in the somatic variants in the biological sample. In some instances, the attributes are arranged from top-to-bottom based on their relative contribution to the identification of the false positives. For example, an attribute corresponding to the top row (“gnomAD_AF”) can be associated with the highest contribution to the identification of the false positives. In this example, gnomAD_AF may refer to a frequency of existing variants in exomes corresponding to a combined population, in which the existing variants are identified from an aggregated genome database (e.g., gnomAD).

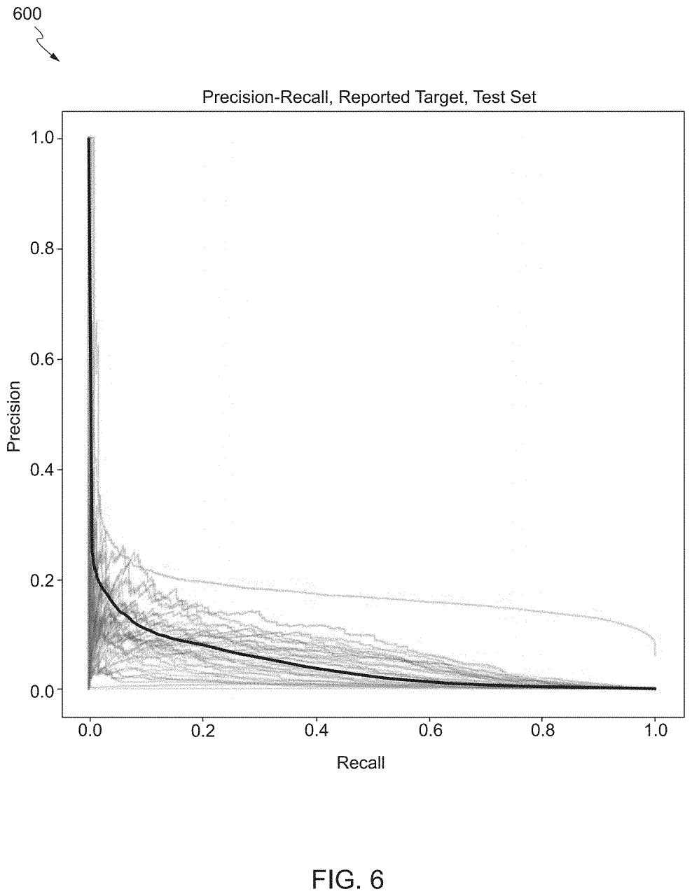

FIG. 6 shows a precision-recall curve 600 corresponding to a trained rescue model for filtering out false negatives from a set of candidate somatic variants, in accordance with some embodiments. As shown in FIG. 6, rescue model data indicates non-linearity in feature importance, and more difficult classification. Due to overwhelming negative class support, precision drops rapidly with increasing recall for the rescue model.

Threshold values for the filtering model 320 can be selected based on maximizing mean sample AUC of the precision recall curve. For example, a threshold value of 0.9995 can be selected for the rescue model 320. In some cases, the rescue model 320 includes a threshold value of at least about 0.1, 0.2, 0.3, 0.4, 0.45, 0.5, 0.55, 0.6, 0.7, 0.8, 0.9, 0.95, 0.96, 0.97, 0.98, 0.99, 0.995, 0.999, 0.9995, 0.9999, or more. In some cases, the rescue model 320 includes a threshold value of at most about 0.3, 0.4, 0.45, 0.5, 0.55, 0.6, 0.7, 0.8, 0.9, 0.95, 0.99, 0.995, 0.999, 0.9995, 0.9999, or less. In some cases, the rescue model 320 includes a threshold value of about 0.2-1.0, 0.2-0.9995, 0.2-0.99, 0.2-0.95, 0.2-0.9, 0.2-0.8, 0.2-0.7, 0.2-0.6, 0.2-0.5, 0.2-0.4, 0.2-0.3, 0.3-1.0, 0.3-0.9995, 0.3-0.99, 0.3-0.95, 0.3-0.9, 0.3-0.8, 0.3-0.7, 0.3-0.6, 0.3-0.5, 0.3-0.4, 0.4-1.0, 0.4-0.9995, 0.4-0.99, 0.4-0.95, 0.4-0.9, 0.4-0.8, 0.4-0.7, 0.4-0.6, 0.4-0.5, 0.5-1.0, 0.5-0.9995, 0.5-0.99, 0.5-0.95, 0.5-0.9, 0.5-0.8, 0.5-0.7, 0.5-0.6, 0.6-1.0, 0.6-0.9995, 0.60.99, 0.6-0.95, 0.6-0.9, 0.6-0.8, 0.6-0.7, 0.7-1.0, 0.7-0.9999, 0.7-0.9995, 0.7-0.999, 0.7-0.99, 0.70.98, 0.7-0.97, 0.7-0.96, 0.7-0.95, 0.7-0.9, 0.7-0.8, 0.8-1.0, 0.8-0.9999, 0.8-0.9995, 0.8-0.999, 0.8-0.99, 0.8-0.98, 0.8-0.97, 0.8-0.96, 0.8-0.95, 0.8-0.9, 0.9-1.0, 0.9-0.9999, 0.9-0.9995, 0.90.999, 0.9-0.99, 0.9-0.98, 0.9-0.97, 0.9-0.96, or 0.9-0.95. In some cases, the rescue model 320 includes a threshold value of about 0.1, 0.2, 0.3, 0.4, 0.45, 0.5, 0.55, 0.6, 0.7, 0.8, 0.9, 0.95, 0.99, 0.995, 0.999, 0.9995, or 0.9999. In some embodiments, the rescue model 320 includes a threshold value of about 0.9 to about 0.9999. In some embodiments, the rescue model 320 includes a threshold value of about 0.9995.

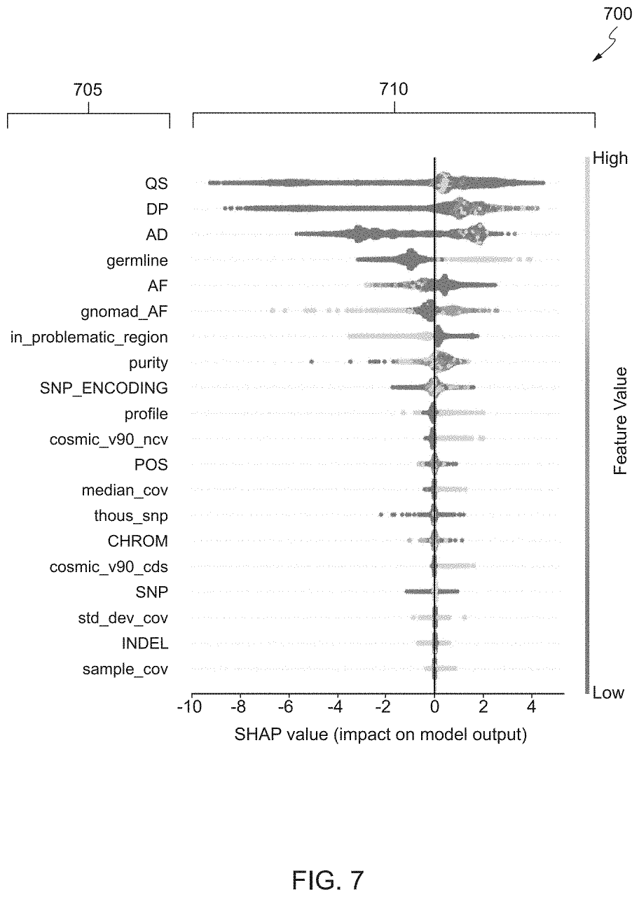

FIG. 7 shows a SHAP plot 700 that identifies which attributes from the attribute table affected the output of a trained rescue model, in accordance with some embodiments. The SHAP plot 700 depicts graphical information that identifies an extent to which each attribute in the attribution table contributed to the identification of false negatives of somatic variants in the biological sample. The SHAP plot 700 includes a left portion 705 that identifies a plurality of attributes derived from the attribute table, in which each row corresponds to one of a plurality of attributes determined for a given candidate variant. The SHAP plot 700 also includes a right portion 710 that identifies, for a given attribute, an extent of contribution to the identification of the false negatives in the somatic variants in the biological sample. In some instances, the attributes are arranged from top-to-bottom based on their relative contribution to the identification of the false negatives. For example, an attribute corresponding to the top row (“QA”) can be associated with the highest contribution to the identification of the false negatives. In this example, QA refers to an alternate allele quality sum in Phred, in which a Phred quality score can indicate a measure of the quality of the identification of the nucleobases generated by automated DNA sequencing.

The ability of the two model classification method to predict somatic variants from unpaired tumor sequencing data can be evaluated before and after training and threshold adjustment. Baseline performance is summarized in TABLE 1. Macro averaged precision and recall statistics are provided for each sample set. Variance is explained by similar true positive rate/false positive rate per sample with varying positive class support.

TABLE 1

| | | Precision | Precision | Recall | Recall |
|---|---|---|---|---|---|
| Method | Data set | mean | SD | mean | SD |
| MuTect | Training | 0.193 | 0.212 | 0.802 | 0.147 |
| MuTect2 | Training | 0.365 | 0.247 | 0.732 | 0.203 |
| Two model | Training | 0.195 | 0.209 | 0.676 | 0.157 |
| classification | | | | | |
| method | | | | | |
| MuTect | Test | 0.159 | 0.148 | 0.808 | 0.154 |
| MuTect2 | Test | 0.32 | 0.218 | 0.745 | 0.177 |
| Two model classification method | Test | 0.161 | 0.147 | 0.685 | 0.156 |

Overall, a precision of 0.189±0.19, and a recall of 0.677±0.15 is observed at baseline. After training and threshold adjustment, the two model classification method achieves a precision of 0.644, with a recall of 0.634.

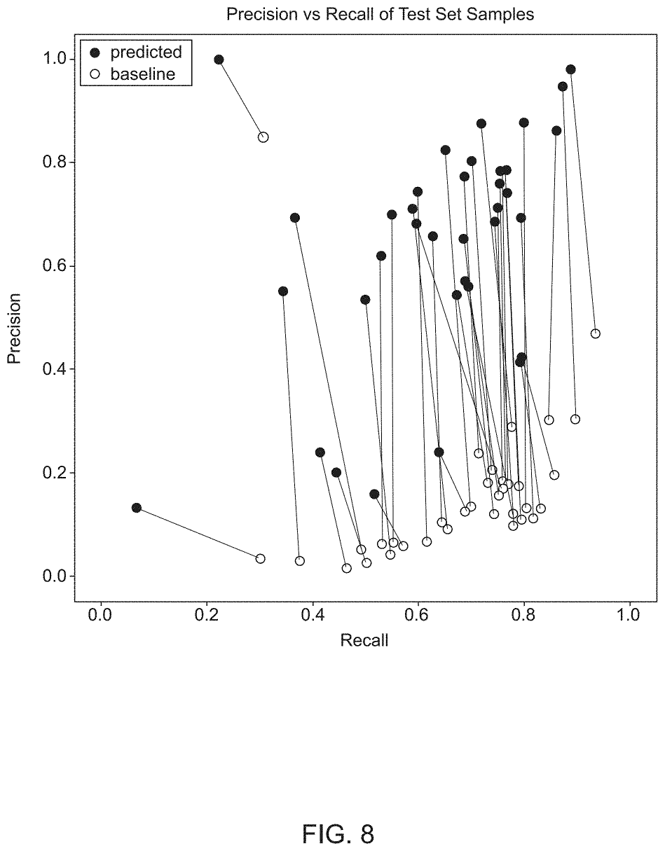

FIG. 8 shows a comparison 800 in the performance of a machine-learning model with a filtering model and a rescue model before and after training and threshold adjustment, in accordance with some embodiments. In this comparison, the machine-learning model was used to predict somatic variants from unpaired tumor sequencing data. Precision and recall values are illustrated at baseline and after training and threshold adjustment.

As shown in FIG. 8, the comparison data indicated that the trained machine-learning model with the filtering model and the rescue model can predict somatic variants from unpaired tumor sequencing data with increased precision compared to alternate methods (e.g., MuTect and MuTect 2).

### III. Identification of Somatic Variants in an Unmatched Biological Sample

A. Subjects and Samples

An unmatched biological sample is obtained from a cancer patient (i.e., a tumor sample without a matching normal sample). The subject can be human. The subject may be a male or a female. The subject may be a fetus, infant, child, adolescent, teenager or adult. The subject may be patients of any age. For example, the subject may be a patient of less than about 10 years old. For example, the subject may be a patient of at least about 0, 5, 10, 20, 30, 40, 50, 60, 70, 80, 90, or 100 years old. Often, the subject is a patient or other individual undergoing a treatment regimen, or being evaluated for a treatment regimen (e.g., cancer therapy). However, in some instances, the subject is not undergoing a treatment regimen.

In some cases, the subjects may be mammals or non-mammals. In some cases, the subjects are a mammal, such as, a human, non-human primate (e.g., apes, monkeys, chimpanzees), cat, dog, rabbit, goat, horse, cow, pig, rodent, mouse, SCID mouse, rat, guinea pig, or sheep. In some methods, species variants or homologs of these genes can be used in a nonhuman animal model. Species variants may be the genes in different species having greatest sequence identity and similarity in functional properties to one another. Many of such species variants human genes may be listed in the Swiss-Prot database.

Some embodiments may include obtaining a sample from a subject, such as a human subject. In particular, the methods may include obtaining a clinical specimen from a patient. For example, blood may be drawn from a patient. Some embodiments may include specifically detecting, profiling, or quantitating molecules (e.g., nucleic acids, DNA, RNA, etc.) that are within the biological samples.

The sample may be a tissue sample or a bodily fluid. In some instances, the sample is a tissue sample or an organ sample, such as a biopsy. In some cases, the sample includes cancerous cells. In some cases, the sample includes cancerous and normal cells. In some cases, the sample is a tumor biopsy. The bodily fluid may be sweat, saliva, tears, urine, blood, menses, semen, and/or spinal fluid. In some cases, the sample is a blood sample. The sample may include one or more peripheral blood lymphocytes. The sample may be a whole blood sample. The blood sample may be a peripheral blood sample. In some cases, the sample includes peripheral blood mononuclear cells (PBMCs); in some cases, the sample includes peripheral blood lymphocytes (PBLs). The sample may be a serum sample.

The sample may be obtained using any method that can provide a sample suitable for the analytical methods described herein. The sample may be obtained by a non-invasive method such as a throat swab, buccal swab, bronchial lavage, urine collection, scraping of the skin or cervix, swabbing of the cheek, saliva collection, feces collection, menses collection, or semen collection. The sample may be obtained by a minimally-invasive method such as a blood draw. The sample may be obtained by venipuncture. In other instances, the sample is obtained by an invasive procedure including but not limited to: biopsy, alveolar or pulmonary lavage, or needle aspiration. The method of biopsy may include surgical biopsy, incisional biopsy, excisional biopsy, punch biopsy, shave biopsy, or skin biopsy. The sample may be formalin fixed sections. The method of needle aspiration may further include fine needle aspiration, core needle biopsy, vacuum assisted biopsy, or large core biopsy. In some cases, multiple samples may be obtained by the methods herein to ensure a sufficient amount of biological material. In some instances, the sample is not obtained by biopsy. In some instances, the sample is not a kidney biopsy.

B. Generating Nucleic Acid Sequencing Data

In some embodiments, the sample is processed to obtain nucleic acid sequence data. “Nucleic acid” or “nucleic acid molecules” can correspond to a polymeric form of nucleotides of any length, either ribonucleotides, deoxyribonucleotides or peptide nucleic acids (PNAs), that include purine and pyrimidine bases, or other natural, chemically or biochemically modified, non-natural, or derivatized nucleotide bases. The backbone of the polynucleotide can include sugars and phosphate groups, as may typically be found in RNA or DNA, or modified or substituted sugar or phosphate groups. A polynucleotide may include modified nucleotides, such as methylated nucleotides and nucleotide analogs. The sequence of nucleotides may be interrupted by non-nucleotide components. Thus, the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecule may be a DNA molecule. The nucleic acid molecule may be an RNA molecule.

DNA is extracted from the tumor sample, processed, and subjected to whole exome sequencing. Sequencing reads are subjected to quality control processing (e.g., via FastQC) to provide FASTQ files. FASTQ files are aligned to a reference genome to generate BAM files.

In some cases, sample processing includes nucleic acid sample processing and subsequent nucleic acid sample sequencing. Some or all of a nucleic acid sample may be sequenced to provide sequence information, which may be stored or otherwise maintained in an electronic, magnetic or optical storage location. The sequence information may be analyzed with the aid of a computer processor, and the analyzed sequence information may be stored in an electronic storage location. The electronic storage location may include a pool or collection of sequence information and analyzed sequence information generated from the nucleic acid sample. The nucleic acid sample may be retrieved from a subject, such as, for example, a subject that has or is suspected of having cancer.

Some embodiments may include using whole genome sequencing. In some cases, the whole genome sequencing is used to identify variants in a person. In some cases, sequencing can include deep sequencing over a fraction of the genome. For example, the fraction of the genome may be at least about 50; 75; 100; 125; 150; 175; 200; 225; 250; 275; 300; 350; 400; 450; 500; 550; 600; 650; 700; 750; 800; 850; 900; 950; 1,000; 1100; 1200; 1300; 1400; 1500; 1600; 1700; 1800; 1900; 2,000; 3,000; 4,000; 5,000; 6,000; 7,000; 8,000; 9,000; 10,000; 15,000; 20,000; 30,000; 40,000; 50,000; 60,000; 70,000; 80,000; 90,000; 100,000 or more bases or base pairs. In some cases, the genome may be sequenced over 1 million, 2 million, 3 million, 4 million, 5 million, 6 million, 7 million, 8 million, 9 million, 10 million or more than 10 million bases or base pairs. In some cases, the genome may be sequenced over an entire exome (e.g., whole exome sequencing). In some cases, the deep sequencing may include acquiring multiple reads over the fraction of the genome. For example, acquiring multiple reads may include at least 2, 3, 4, 5, 6, 7, 8, 9, 10, 20, 30, 40, 50, 60, 70, 80, 90, 100, 200, 300, 400, 500, 600, 700, 800, 900, 1000, 10,000 reads or more than 10,000 reads over the fraction of the genome.

Some embodiments may include detecting low allelic fractions by deep sequencing. In some cases, the deep sequencing is done by next generation sequencing. In some cases, the deep sequencing is done by avoiding error-prone regions. In some cases, the error-prone regions may include regions of near sequence duplication, regions of unusually high or low % GC, regions of near homopolymers, di- and tri-nucleotide, and regions of near other short repeats. In some cases, the error-prone regions may include regions that lead to DNA sequencing errors (e.g., polymerase slippage in homopolymer sequences).

Some embodiments may include conducting one or more sequencing reactions on one or more nucleic acid molecules in a sample. Some embodiments may include conducting 1 or more, 2 or more, 3 or more, 4 or more, 5 or more, 6 or more, 7 or more, 8 or more, 9 or more, 10 or more, 15 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more sequencing reactions on one or more nucleic acid molecules in a sample. The sequencing reactions may be run simultaneously, sequentially, or a combination thereof. The sequencing reactions may include whole genome sequencing or exome sequencing. The sequencing reactions may include Maxim-Gilbert, chain-termination or high-throughput systems. Alternatively, or additionally, the sequencing reactions may include Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT™ sequencing, Polony sequencing, DNA nanoball sequencing, VisiGen Biotechnologies approach, or a combination thereof. Alternatively, or additionally, the sequencing reactions can include one or more sequencing platforms, including, but not limited to, Genome Analyzer IIx, HiSeq, and MiSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, Mass.). Sequencing reactions may also include electron microscopy or a chemical-sensitive field effect transistor (chemFET) array. In some aspects, sequencing reactions include capillary sequencing, next generation sequencing, Sanger sequencing, sequencing by synthesis, sequencing by ligation, sequencing by hybridization, single molecule sequencing, or a combination thereof. Sequencing by synthesis may include reversible terminator sequencing, processive single molecule sequencing, sequential flow sequencing, or a combination thereof. Sequential flow sequencing may include pyrosequencing, pH-mediated sequencing, semiconductor sequencing, or a combination thereof.

Some embodiments may include conducting at least one long read sequencing reaction and at least one short read sequencing reaction. The long read sequencing reaction and/or short read sequencing reaction may be conducted on at least a portion of a subset of nucleic acid molecules. The long read sequencing reaction and/or short read sequencing reaction may be conducted on at least a portion of two or more subsets of nucleic acid molecules. Both a long read sequencing reaction and a short read sequencing reaction may be conducted on at least a portion of one or more subsets of nucleic acid molecules.

Sequencing of the one or more nucleic acid molecules or subsets thereof may include at least about 5; 10; 15; 20; 25; 30; 35; 40; 45; 50; 60; 70; 80; 90; 100; 200; 300; 400; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 25,000; 50,000; 75,000; 100,000; 250,000; 500,000; 750,000; 10,000,000; 25,000,000; 50,000,000; 100,000,000; 250,000,000; 500,000,000; 750,000,000; 1,000,000,000 or more sequencing reads.

Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 20,000; 30,000; 40,000; 50,000; 60,000; 70,000; 80,000; 90,000; 100,000 or more bases or base pairs of one or more nucleic acid molecules. Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 20,000; 30,000; 40,000; 50,000; 60,000; 70,000; 80,000; 90,000; 100,000 or more consecutive bases or base pairs of one or more nucleic acid molecules.

Preferably, the sequencing techniques used in the methods of the invention generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, or at least 1,000,000 reads per run. Alternatively, the sequencing technique used in the methods of the invention generates at least 1,500,000 reads per run, at least 2,000,000 reads per run, at least 2,500,000 reads per run, at least 3,000,000 reads per run, at least 3,500,000 reads per run, at least 4,000,000 reads per run, at least 4,500,000 reads per run, or at least 5,000,000 reads per run.

Preferably, the sequencing techniques used in the methods of the invention can generate at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110, at least about 120 base pairs per read, at least about 150 base pairs, at least about 200 base pairs, at least about 250 base pairs, at least about 300 base pairs, at least about 350 base pairs, at least about 400 base pairs, at least about 450 base pairs, at least about 500 base pairs, at least about 550 base pairs, at least about 600 base pairs, at least about 700 base pairs, at least about 800 base pairs, at least about 900 base pairs, or at least about 1,000 base pairs per read. Alternatively, the sequencing technique used in the methods of the invention can generate long sequencing reads. In some instances, the sequencing technique used in the methods of the invention can generate at least about 1,200 base pairs per read, at least about 1,500 base pairs per read, at least about 1,800 base pairs per read, at least about 2,000 base pairs per read, at least about 2,500 base pairs per read, at least about 3,000 base pairs per read, at least about 3,500 base pairs per read, at least about 4,000 base pairs per read, at least about 4,500 base pairs per read, at least about 5,000 base pairs per read, at least about 6,000 base pairs per read, at least about 7,000 base pairs per read, at least about 8,000 base pairs per read, at least about 9,000 base pairs per read, at least about 10,000 base pairs per read, 20,000 base pairs per read, 30,000 base pairs per read, 40,000 base pairs per read, 50,000 base pairs per read, 60,000 base pairs per read, 70,000 base pairs per read, 80,000 base pairs per read, 90,000 base pairs per read, or 100,000 base pairs per read.

High-throughput sequencing systems may allow detection of a sequenced nucleotide immediately after or upon its incorporation into a growing strand, i.e., detection of sequence in real time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120, at least 150, at least 200, at least 250, at least 300, at least 350, at least 400, at least 450, or at least 500 bases per read. Sequencing can be performed using nucleic acids described herein such as genomic DNA, cDNA derived from RNA transcripts or RNA as a template.

C. Identifying Candidate Variants

The nucleic acid sequence data can be aligned to a reference genome. Based on the aligned nucleic acid sequence data, a set of candidate variants in the nucleic acid sequence data can be identified. In some instances, the set of candidate variants includes one or more somatic variants and one or more germline variants. For example, BCFtools can be used to identify a set of candidate somatic variants for each sample at high sensitivity. The set of candidate somatic variants will include false positives, e.g., germline variants.

For the set of candidate somatic variants, an attribute table is generated including a number of features for each candidate variant (e.g., about 10-20 features). The attribute table can include any combination of the attributes described in example 3. The attribute table can include a number of features for each candidate variant. Examples of features the attribute table can contain, include, but are not limited to, (i) pileup attributes from the initial BCFtools output, such as allelic frequency (e.g., B allele frequency), base quality, read depth, etc.; (ii) an estimate of tumor purity determined using a deep learning neural network, based on whole exome B allele frequency distribution in the sample; (iii) whether the variant is identified as a germline variant using GATK HaplotypeCaller; (iv) somatic copy number alteration (CNA) state for each variant site; (v) the frequency of the variant in populations (e.g., in healthy human populations and/or in cancer exomes from databases such as Cosmic, GnomAD, Dbsnp, Mills Indels, etc.); (vi) presence of the variant in problematic regions, such as in homopolymers; and (vii) whether the variant is identified by standard somatic callers (run in the single-tumor context), e.g., MuTect and MuTect2.

An attribute table can include any number of features that can contribute to accurate prediction of somatic variants. For example, an attribute table can include at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 features, or more. In some cases, an attribute table can include at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 features, or less. In some embodiments, an attribute table can include about 1-100, 1-90, 1-80, 1-70, 1-60, 1-50, 1-40, 1-30, 1-20, 1-10, 1-5, 5-100, 5-90, 580, 5-70, 5-60, 5-50, 5-40, 5-30, 5-20, 5-10, 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, or 20-30 features. In some cases, an attribute table includes about 10 to 20 features.

In some embodiments, identifying the set of candidate variants may include identifying one or more genomic regions that include one or more nucleotide-sequence variants. The one or more genomic regions may include one or more genomic region features. The genomic region features may include an entire genome or a portion thereof. The genomic region features may include an entire exome or a portion thereof. The genomic region features may include one or more sets of genes. The genomic region features may include one or more genes. The genomic region features may include one or more sets of regulatory elements. The genomic region features may include one or more regulatory elements. The genomic region features may include a set of polymorphisms. The genomic region features may include one or more polymorphisms. The genomic region feature may relate to the GC content, complexity, and/or mappability of one or more nucleic acid molecules. The genomic region features may include one or more simple tandem repeats (STRs), unstable expanding repeats, segmental duplications, single and paired read degenerative mapping scores, GRCh37 patches, or a combination thereof. The genomic region features may include one or more low mean coverage regions from whole genome sequencing (WGS), zero mean coverage regions from WGS, validated compressions, or a combination thereof. The genomic region features may include one or more alternate or non-reference sequences. The genomic region features may include one or more gene phasing and reassembly genes. In some aspects, the one or more genomic region features are not mutually exclusive. For example, a genomic region feature including an entire genome or a portion thereof can overlap with an additional genomic region feature such as an entire exome or a portion thereof, one or more genes, one or more regulatory elements, etc. Alternatively, the one or more genomic region futures are mutually exclusive. For example, a genomic region including the noncoding portion of an entire genome would not overlap with a genomic region feature such as an exome or portion thereof or the coding portion of a gene. Alternatively, or additionally, the one or more genomic region features are partially exclusive or partially inclusive. For example, a genomic region including an entire exome or a portion thereof can partially overlap with a genomic region including an exon portion of a gene. However, the genomic region including the entire exome or portion thereof would not overlap with the genomic region including the intron portion of the gene. Thus, a genomic region feature including a gene or portion thereof may partially exclude and/or partially include a genomic region feature including an entire exome or portion thereof.

Some embodiments may include nucleic acid samples or molecules including one or more genomic regions, wherein at least one of the one or more genomic regions includes a genomic region feature including an entire genome or portion thereof. The entire genome or portion thereof may include one or more coding portions of the genome, one or more noncoding portions of the genome, or a combination thereof. The coding portion of the genome may include one or more coding portions of a gene encoding for one or more proteins. The one or more coding portions of the genome may include an entire exome or a portion thereof. Alternatively, or additionally, the one or more coding portions of the genome may include one or more exons. The one or more noncoding portions of the genome may include one or more noncoding molecules or a portion thereof. The noncoding molecules may include one or more noncoding RNA, one or more regulatory elements, one or more introns, one or more pseudogenes, one or more repeat sequences, one or more transposons, one or more viral elements, one or more telomeres, a portion thereof, or a combination thereof. The noncoding RNAs may be functional RNA molecules that are not translated into protein. Examples of noncoding RNAs include, but are not limited to, ribosomal RNA, transfer RNA, piwi-interacting RNA, microRNA, siRNA, shRNA, snoRNA, sncRNA, and lncRNA. Pseudogenes may be related to known genes and are typically no longer expressed. Repeat sequences may include one or more tandem repeats, one or more interspersed repeats, or a combination thereof. Tandem repeats may include one or more satellite DNA, one or more minisatellites, one or more microsatellites, or a combination thereof. Interspersed repeats may include one or more transposons. Transposons may be mobile genetic elements. Mobile genetic elements are often able to change their position within the genome. Transposons may be classified as class I transposable elements (class I TEs) or class II transposable elements (class II TEs). Class I TEs (e.g., retrotransposons) may often copy themselves in two stages, first from DNA to RNA by transcription, then from RNA back to DNA by reverse transcription. The DNA copy may then be inserted into the genome in a new position. Class I TEs may include one or more long terminal repeats (LTRs), one or more long interspersed nuclear elements (LINEs), one or more short interspersed nuclear elements (SINEs), or a combination thereof. Examples of LTRs include, but are not limited to, human endogeneous retroviruses (HERV5), medium reiterated repeats 4 (MER4), and retrotransposon. Examples of LINEs include, but are not limited to, LINE1 and LINE2. SINEs may include one or more Alu sequences, one or more mammalian-wide interspersed repeat (MIR), or a combination thereof. Class II TEs (e.g., DNA transposons) often do not involve an RNA intermediate. The DNA transposon is often cut from one site and inserted into another site in the genome. Alternatively, the DNA transposon is replicated and inserted into the genome in a new position. Examples of DNA transposons include, but are not limited to, MER1, MER2, and mariners. Viral elements may include one or more endogenous retrovirus sequences. Telomeres are often regions of repetitive DNA at the end of a chromosome.

Some embodiments may include nucleic acid samples or subsets of nucleic acid molecules including one or more genomic regions, wherein at least one of the one or more genomic regions includes a genomic region feature including an entire exome or portion thereof. The exome is often the part of the genome formed by exons. The exome may be formed by untranslated regions (UTRs), splice sites and/or intronic regions. The entire exome or portion thereof may include one or more exons of a protein coding gene. The entire exome or portion thereof may include one or more untranslated regions (UTRs), splice sites, and introns.

Some embodiments may include nucleic acid samples or molecules including one or more genomic regions, wherein at least one of the one or more genomic regions includes a genomic region feature including a gene or portion thereof. Typically, a gene includes stretches of nucleic acids that code for a polypeptide or a functional RNA. A gene may include one or more exons, one or more introns, one or more untranslated regions (UTRs), or a combination thereof. Exons are often coding sections of a gene, transcribed into a precursor mRNA sequence, and within the final mature RNA product of the gene. Introns are often noncoding sections of a gene, transcribed into a precursor mRNA sequence, and removed by RNA splicing. UTRs may refer to sections on each side of a coding sequence on a strand of mRNA. A UTR located on the 5′ side of a coding sequence may be called the 5′ UTR (or leader sequence). A UTR located on the 3′ side of a coding sequence may be called the 3′ UTR (or trailer sequence). The UTR may contain one or more elements for controlling gene expression. Elements, such as regulatory elements, may be located in the 5′ UTR. Regulatory sequences, such as a polyadenylation signal, binding sites for proteins, and binding sites for miRNAs, may be located in the 3′ UTR. Binding sites for proteins located in the 3′ UTR may include, but are not limited to, selenocysteine insertion sequence (SECIS) elements and AU-rich elements (AREs). SECIS elements may direct a ribosome to translate the codon UGA as selenocysteine rather than as a stop codon. AREs are often stretches consisting primarily of adenine and uracil nucleotides, which may affect the stability of a mRNA.

Some embodiments may include nucleic acid samples or subsets of nucleic acid molecules including one or more genomic regions, wherein at least one of the one or more genomic regions includes a genomic region feature including a set of genes. The sets of genes may include, but are not limited to, Mendel DB Genes, Human Gene Mutation Database (HGMD) Genes, Cancer Gene Census Genes, Online Mendelian Inheritance in Man (OMIM) Mendelian Genes, HGMD Mendelian Genes, and human leukocyte antigen (HLA) Genes. The set of genes may have one or more known Mendelian traits, one or more known disease traits, one or more known drug traits, one or more known biomedically interpretable variants, or a combination thereof. A Mendelian trait may be controlled by a single locus and may show a Mendelian inheritance pattern. A set of genes with known Mendelian traits may include one or more genes encoding Mendelian traits including, but are not limited to, ability to taste phenylthiocarbamide (dominant), ability to smell (bitter almond-like) hydrogen cyanide (recessive), albinism (recessive), brachydactyly (shortness of fingers and toes), and wet (dominant) or dry (recessive) earwax. A disease trait cause or increase risk of disease and may be inherited in a Mendelian or complex pattern. A set of genes with known disease traits may include one or more genes encoding disease traits including, but are not limited to, Cystic Fibrosis, Hemophilia, and Lynch Syndrome. A drug trait may alter metabolism, optimal dose, adverse reactions and side effects of one or more drugs or family of drugs. A set of genes with known drug traits may include one or more genes encoding drug traits including, but are not limited to, CYP2D6, UGT1A1 and ADRB1. A biomedically interpretable variant may be a polymorphism in a gene that is associated with a disease or indication. A set of genes with known biomedically interpretable variants may include one or more genes encoding biomedically interpretable variants including, but are not limited to, cystic fibrosis (CF) mutations, muscular dystrophy mutations, p53 mutations, Rb mutations, cell cycle regulators, receptors, and kinases. Alternatively, or additionally, a set of genes with known biomedically interpretable variants may include one or more genes associated with Huntington's disease, cancer, cystic fibrosis, muscular dystrophy (e.g., Duchenne muscular dystrophy).

Some embodiments may include nucleic acid samples or molecules including one or more genomic regions, wherein at least one of the one or more genomic regions includes a genomic region feature including a regulatory element or a portion thereof. Regulatory elements may be cis-regulatory elements or trans-regulatory elements. Cis-regulatory elements may be sequences that control transcription of a nearby gene. Cis-regulatory elements may be located in the 5′ or 3′ untranslated regions (UTRs) or within introns. Trans-regulatory elements may control transcription of a distant gene. Regulatory elements may include one or more promoters, one or more enhancers, or a combination thereof. Promoters may facilitate transcription of a particular gene and may be found upstream of a coding region. Enhancers may exert distant effects on the transcription level of a gene.

Some embodiments may include nucleic acid samples or subsets of nucleic acid molecules including one or more genomic regions, wherein at least one of the one or more genomic regions includes a genomic region feature including a polymorphism or a portion thereof. Generally, a polymorphism refers to a mutation in a genotype. A polymorphism can be a germline variant or a somatic variant. A polymorphism may include one or more base changes, an insertion, a repeat, or a deletion of one or more bases. Copy number variants (CNVs), transversions and other rearrangements are also forms of genetic variation. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are a form of polymorphisms. In some aspects, one or more polymorphisms include one or more single nucleotide variations, inDels, small insertions, small deletions, structural variant junctions, variable length tandem repeats, flanking sequences, or a combination thereof. The one or more polymorphisms may be located within a coding and/or noncoding region. The one or more polymorphisms may be located within, around, or near a gene, exon, intron, splice site, untranslated region, or a combination thereof. The one or more polymorphisms may be may span at least a portion of a gene, exon, intron, untranslated region.

Some embodiments may include nucleic acid samples or molecules including one or more genomic regions, wherein at least one of the one or more genomic regions includes a genomic region feature including one or more simple tandem repeats (STRs), unstable expanding repeats, segmental duplications, single and paired read degenerative mapping scores, GRCh37 patches, or a combination thereof. The one or more STRs may include one or more homopolymers, one or more dinucleotide repeats, one or more trinucleotide repeats, or a combination thereof. The one or more homopolymers may be about 7, 8, 9, 10, 11, 12, 13, 14, 15, 16, 17, 18, 19, 20 or more bases or base pairs. The dinucleotide repeats and/or trinucleotide repeats may be about 15, 16, 17, 18, 19, 20, 21, 22, 23, 24, 25, 26, 27, 28, 29, 30, 35, 40, 45, 50 or more bases or base pairs. The single and paired read degenerative mapping scores may be based on or derived from alignability of 100 mers by GEM from ENCODE/CRG (Guigo), alignability of 75 mers by GEM from ENCODE/CRG (Guigo), 100 base pair box car average for signal mappability, max of locus and possible pairs for paired read score, or a combination thereof. The genomic region features may include one or more low mean coverage regions from whole genome sequencing (WGS), zero mean coverage regions from WGS, validated compressions, or a combination thereof. The low mean coverage regions from WGS may include regions generated from Illumina v3 chemistry, regions below the first percentile of Poission distribution based on mean coverage, or a combination thereof. The Zero mean coverage regions from WGS may include regions generated from Illumina v3 chemistry. The validated compressions may include regions of high mapped depth, regions with two or more observed haplotypes, regions expected to be missing repeats in a reference, or a combination thereof. The genomic region features may include one or more alternate or non-reference sequences. The one or more alternate or non-reference sequences may include known structural variant junctions, known insertions, known deletions, alternate haplotypes, or a combination thereof. The genomic region features may include one or more gene phasing and reassembly genes. Examples of phasing and reassembly genes include, but are not limited to, one or more major histocompatibility complexes, blood typing, and amylase gene family. The one or more major histocompatibility complexes may include one or more HLA Class I, HLA Class II, or a combination thereof. The one or more HLA class I may include HLA-A, HLA-B, HLA-C, or a combination thereof. The one or more HLA class II may include HLA-DP, HLA-DM, HLA-DOA, HLA-DOB, HLA-DQ, HLA-DR, or a combination thereof. The blood typing genes may include ABO, RHD, RHCE, or a combination thereof.

Some embodiments may include nucleic acid samples or molecules including one or more genomic regions, wherein at least one of the one or more genomic regions includes a genomic region feature related to the GC content of one or more nucleic acid molecules. The GC content may refer to the GC content of a nucleic acid molecule. Alternatively, the GC content may refer to the GC content of one or more nucleic acid molecules and may be referred to as the mean GC content. As used herein, the terms “GC content” and “mean GC content” may be used interchangeably. The GC content of a genomic region may be a high GC content. Typically, a high GC content refers to a GC content of greater than or equal to about 65%, 70%, 75%, 80%, 85%, 90%, 95%, 97%, or more. In some aspects, a high GC content may refer to a GC content of greater than or equal to about 70%. The GC content of a genomic region may be a low GC content. Typically, a low GC content refers to a GC content of less than or equal to about 65%, 60%, 55%, 50%, 45%, 40%, 35%, 30%, 25%, 20%, 15%, 10%, 5%, 2%, or less.

Some embodiments may include nucleic acid samples or molecules including one or more genomic regions, wherein at least one of the one or more genomic regions includes a genomic region feature related to the complexity of one or more nucleic acid molecules. The complexity of a nucleic acid molecule may refer to the randomness of a nucleotide sequence. Low complexity may refer to patterns, repeats and/or depletion of one or more species of nucleotide in the sequence.

Some embodiments may include nucleic acid samples or molecules including one or more genomic regions, wherein at least one of the one or more genomic regions includes a genomic region feature related to the mappability of one or more nucleic acid molecules. The mappability of a nucleic acid molecule may refer to uniqueness of its alignment to a reference sequence. A nucleic acid molecule with low mappability may have poor alignment to a reference sequence.

D. Predicting Whether a Candidate Variant is a Somatic Variant

A two model classification method is used to predict somatic variants from the attribute table. For example, the attribute table can subdivided into two data sets as illustrated in FIG. 4 and processed using trained models, for example the models described in example 3. The first dataset can contain candidate somatic variants identified by one or more bioinformatic tools. A first model can be applied to filter false positives out of this dataset. The second dataset can contain the remainder of candidate variants, including false negatives and true negatives. A second model can be applied to rescue false negatives from this dataset. The method can predict somatic variants with acceptable accuracy despite the lack of a matching normal sample.

For increased control of thresholding, the somatic variant classification problem is decomposed into two sub-problems: (1) filter out false positives in tumor-only calls from each variant caller, and (2) rescue false negative candidate variants not present in tumor-only calls. The attribute table is subdivided into two datasets. The first dataset contains candidate variants that are identified by MuTect and MuTect2 (in the tumor-only context). A first model is trained to filter false positives out of this dataset. The second dataset contains the remainder of candidate variants. A second model is trained to rescue false negatives from this dataset. The models are trained using Microsoft's LightGBM framework (LGBM). Classification results from both of these models are then combined to produce a final set of somatic variants.

E. Generating a Report Identifying the Somatic Variants

One or more reports can be generated that include some or all of the predicted somatic variants (e.g., diagnostic and/or prognostic reports). One or more treatments can be administered to the patient or withheld from the patient based on the predicted somatic variants and/or the report(s). For example, the predicted somatic variants can be compared to one or more databases of known cancer mutations to diagnose or characterize the cancer. Variants can be identified that are associated with responsiveness or unresponsiveness to certain cancer treatments, and a treatment recommendation can be provided. The cancer can be treated based on the recommendation.

### IV. Process for Somatic Variant Calling from Unmatched Biological Samples

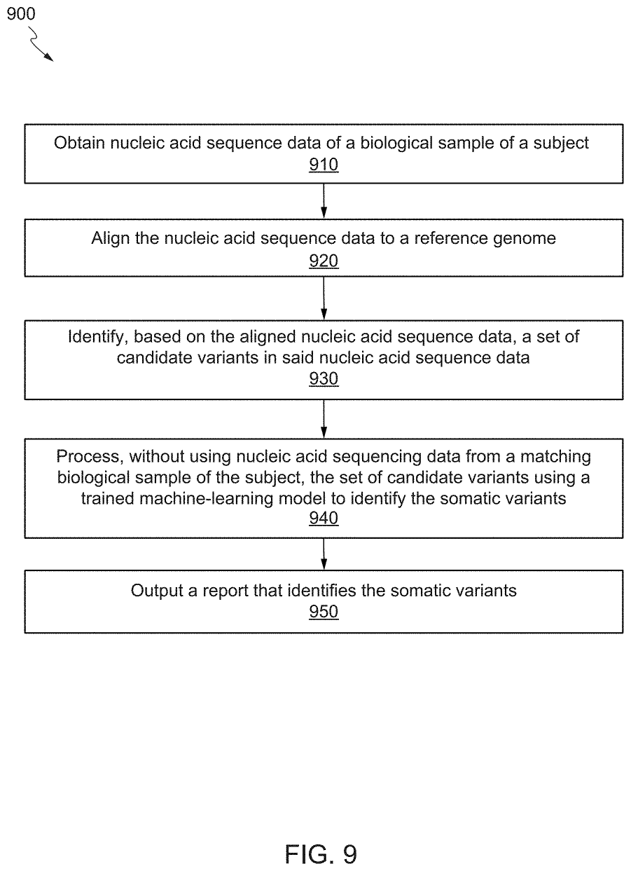

FIG. 9 includes a flowchart 900 illustrating an example of a method of somatic variant calling from unmatched biological samples according to some embodiments. Operations described in flowchart 900 may be performed by, for example, a computer system implementing a trained machine-learning model that includes a filtering model and a rescue model. Although flowchart 900 may describe the operations as a sequential process, in various embodiments, many of the operations may be performed in parallel or concurrently. In addition, the order of the operations may be rearranged. An operation may have additional steps not shown in the figure. Furthermore, embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At operation 910, a computer system obtains nucleic acid sequence data a biological sample of a subject. The nucleic acid sequence data can be generated by sequencing the plurality of nucleic acid molecules of the tumor sample. In some embodiments, the tumor sample is from a human subject. Sequencing can include whole exome sequencing. In some embodiments, the sequencing can include whole genome sequencing. In some embodiments, the sequencing includes shotgun sequencing. In some embodiments, the sequencing includes sequencing select parts of the genome or exome.

At operation 920, the computer system aligns the nucleic acid sequence data to a reference genome. For example, the FASTQ files, which correspond to the nucleic acid sequence data, can be aligned to a reference genome to generate one or more BAM files.

At operation 930, the computer system identifies, based on the aligned nucleic acid sequence data, a set of candidate variants in said nucleic acid sequence data. In some instances, the set of candidate variants includes one or more somatic variants and one or more germline variants. The somatic variants refer to an alteration in DNA that occurs after conception and is not present within the germline. The germline variants refer to a gene change in a reproductive cell (egg or sperm) that becomes incorporated into the DNA of every cell in the body of the offspring. In some instances, the somatic variants, instead of the germline variants, indicate a presence or a level of cancer in the subject.

An attribute table can be generated, in which the attribute table can include a number of features for each candidate variant. In some embodiments, the attribute table includes attributes from sequencing data that corresponds to a particular candidate variant. The attribute table can include attributes from a file including processed sequencing data. In some embodiments, the attribute table includes one or more attributes as follows: (a) pileup attributes from a BCFtools output file; (b) allelic frequency data; (c) base quality data; (d) read depth data; (e) an estimation of tumor cellularity (which may be calculated based on a B allele frequency distribution); (f) predicted germline variants; (g) predicted somatic variants; (h) copy number alteration data; (i) population frequency data from one or more databases; (j) data from at least one database selected from the group consisting of Cosmic, GnomAD, Dbsnp, and Mills Indels; (k) data regarding the presence of candidate somatic variants in problematic regions of the genome; and (1) data regarding the presence of candidate somatic variants in homopolymers.

At operation 940, the computer system processes, without using nucleic acid sequencing data from a matching biological sample of the subject, the set of candidate variants using a trained machine-learning model to identify the somatic variants. In some instances, the trained machine-learning model includes gradient-boosted decision trees that facilitate significant reduction of false positive rate corresponding to somatic-variant calls. In some embodiments, the trained machine-learning model includes a two model classification method. The trained machine-learning model may include a filtration model that filters out false positives. The trained machine-learning model may also include a rescue model that rescues false negatives. In some embodiments, the attribute table includes attributes from the sequencing data.

At operation 950, the computer system outputs a report that identifies the somatic variants. In some embodiments, the report includes information identifying at least one diagnostic marker, at least one prognostic marker. In some embodiments, an absence of a somatic variant, a treatment recommendation, a recommendation to administer a treatment to the human subject, and/or a recommendation to not administer a treatment to the human subject. In some embodiments, the recommended treatment is administered to the human subject. Process 900 terminates thereafter.

### V. Additional Considerations

A. Probing Techniques

Some embodiments may include one or more labels. The one or more labels may be attached to one or more capture probes, nucleic acid molecules, beads, primers, or a combination thereof. Examples of labels include, but are not limited to, detectable labels, such as radioisotopes, fluorophores, chemiluminophores, chromophore, lumiphore, enzymes, colloidal particles, and fluorescent microparticles, quantum dots, as well as antigens, antibodies, haptens, avidin/streptavidin, biotin, haptens, enzymes cofactors/substrates, one or more members of a quenching system, a chromogens, haptens, a magnetic particles, materials exhibiting nonlinear optics, semiconductor nanocrystals, metal nanoparticles, enzymes, aptamers, and one or more members of a binding pair.

Some embodiments may include one or more capture probes, a plurality of capture probes, or one or more capture probe sets. Typically, the capture probe includes a nucleic acid binding site. The capture probe may further include one or more linkers. The capture probes may further include one or more labels. The one or more linkers may attach the one or more labels to the nucleic acid binding site.

Capture probes may hybridize to one or more nucleic acid molecules in a sample. Capture probes may hybridize to one or more genomic regions. Capture probes may hybridize to one or more genomic regions within, around, near, or spanning one or more genes, exons, introns, UTRs, or a combination thereof. Capture probes may hybridize to one or more genomic regions spanning one or more genes, exons, introns, UTRs, or a combination thereof. Capture probes may hybridize to one or more known inDels. Capture probes may hybridize to one or more known structural variants.

Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more capture probes or capture probe sets. The one or more capture probes or capture probe sets may be different, similar, identical, or a combination thereof.

The one or more capture probe may include a nucleic acid binding site that hybridizes to at least a portion of the one or more nucleic acid molecules or variant or derivative thereof in the sample or subset of nucleic acid molecules. The capture probes may include a nucleic acid binding site that hybridizes to one or more genomic regions. The capture probes may hybridize to different, similar, and/or identical genomic regions. The one or more capture probes may be at least about 50%, 55%, 60%, 65%, 70%, 75%, 80%, 85%, 90%, 95%, 97%, 99% or more complementary to the one or more nucleic acid molecules or variant or derivative thereof.

The capture probes may include one or more nucleotides. The capture probes may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more nucleotides. The capture probes may include about 100 nucleotides. The capture probes may include between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects, the capture probes include between about 80 nucleotides to about 100 nucleotides.

The plurality of capture probes or the capture probe sets may include two or more capture probes with identical, similar, and/or different nucleic acid binding site sequences, linkers, and/or labels. For example, two or more capture probes include identical nucleic acid binding sites. In another example, two or more capture probes include similar nucleic acid binding sites. In yet another example, two or more capture probes include different nucleic acid binding sites. The two or more capture probes may further include one or more linkers. The two or more capture probes may further include different linkers. The two or more capture probes may further include similar linkers. The two or more capture probes may further include identical linkers. The two or more capture probes may further include one or more labels. The two or more capture probes may further include different labels. The two or more capture probes may further include similar labels. The two or more capture probes may further include identical labels.

B. Assays and Amplification Techniques

Some embodiments may include conducting one or more assays on a sample including one or more nucleic acid molecules. Producing two or more subsets of nucleic acid molecules may include conducting one or more assays. The assays may be conducted on a subset of nucleic acid molecules from the sample. The assays maybe conducted on one or more nucleic acids molecules from the sample. The assays may be conducted on at least a portion of a subset of nucleic acid molecules. The assays may include one or more techniques, reagents, capture probes, primers, labels, and/or components for the detection, quantification, and/or analysis of one or more nucleic acid molecules.

Assays may include, but are not limited to, sequencing, amplification, hybridization, enrichment, isolation, elution, fragmentation, detection, quantification of one or more nucleic acid molecules. Assays may include methods for preparing one or more nucleic acid molecules.

Some embodiments may include conducting one or more amplification reactions on one or more nucleic acid molecules in a sample. The term “amplification” refers to any process of producing at least one copy of a nucleic acid molecule. The terms “amplicons” and “amplified nucleic acid molecule” refer to a copy of a nucleic acid molecule and can be used interchangeably. The amplification reactions can include PCR-based methods, non-PCR based methods, or a combination thereof. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. PCR-based methods may include, but are not limited to, PCR, HD-PCR, Next Gen PCR, digital RTA, or any combination thereof. Additional PCR methods include, but are not limited to, linear amplification, allele-specific PCR, Alu PCR, assembly PCR, asymmetric PCR, droplet PCR, emulsion PCR, helicase dependent amplification HDA, hot start PCR, inverse PCR, linear-after-the-exponential (LATE)-PCR, long PCR, multiplex PCR, nested PCR, hemi-nested PCR, quantitative PCR, RT-PCR, real time PCR, single cell PCR, and touchdown PCR.

Some embodiments may include conducting one or more hybridization reactions on one or more nucleic acid molecules in a sample. The hybridization reactions may include the hybridization of one or more capture probes to one or more nucleic acid molecules in a sample or subset of nucleic acid molecules. The hybridization reactions may include hybridizing one or more capture probe sets to one or more nucleic acid molecules in a sample or subset of nucleic acid molecules. The hybridization reactions may include one or more hybridization arrays, multiplex hybridization reactions, hybridization chain reactions, isothermal hybridization reactions, nucleic acid hybridization reactions, or a combination thereof. The one or more hybridization arrays may include hybridization array genotyping, hybridization array proportional sensing, DNA hybridization arrays, macroarrays, microarrays, high-density oligonucleotide arrays, genomic hybridization arrays, comparative hybridization arrays, or a combination thereof. The hybridization reaction may include one or more capture probes, one or more beads, one or more labels, one or more subsets of nucleic acid molecules, one or more nucleic acid samples, one or more reagents, one or more wash buffers, one or more elution buffers, one or more hybridization buffers, one or more hybridization chambers, one or more incubators, one or more separators, or a combination thereof.

Some embodiments may include conducting one or more enrichment reactions on one or more nucleic acid molecules in a sample. The enrichment reactions may include contacting a sample with one or more beads or bead sets. The enrichment reaction may include differential amplification of two or more subsets of nucleic acid molecules based on one or more genomic region features. For example, the enrichment reaction includes differential amplification of two or more subsets of nucleic acid molecules based on GC content. Alternatively, or additionally, the enrichment reaction includes differential amplification of two or more subsets of nucleic acid molecules based on methylation state. The enrichment reactions may include one or more hybridization reactions. The enrichment reactions may further include isolation and/or purification of one or more hybridized nucleic acid molecules, one or more bead bound nucleic acid molecules, one or more free nucleic acid molecules (e.g., capture probe free nucleic acid molecules, bead free nucleic acid molecules), one or more labeled nucleic acid molecules, one or more non-labeled nucleic acid molecules, one or more amplicons, one or more non-amplified nucleic acid molecules, or a combination thereof. Alternatively, or additionally, the enrichment reaction may include enriching for one or more cell types in the sample. The one or more cell types may be enriched by flow cytometry.

The one or more enrichment reactions may produce one or more enriched nucleic acid molecules. The enriched nucleic acid molecules may include a nucleic acid molecule or variant or derivative thereof. For example, the enriched nucleic acid molecules include one or more hybridized nucleic acid molecules, one or more bead bound nucleic acid molecules, one or more free nucleic acid molecules (e.g., capture probe free nucleic acid molecules, bead free nucleic acid molecules), one or more labeled nucleic acid molecules, one or more non-labeled nucleic acid molecules, one or more amplicons, one or more non-amplified nucleic acid molecules, or a combination thereof. The enriched nucleic acid molecules may be differentiated from non-enriched nucleic acid molecules by GC content, molecular size, genomic regions, genomic region features, or a combination thereof. The enriched nucleic acid molecules may be derived from one or more assays, supernatants, eluants, or a combination thereof. The enriched nucleic acid molecules may differ from the non-enriched nucleic acid molecules by mean size, mean GC content, genomic regions, or a combination thereof.

Some embodiments may include conducting one or more isolation or purification reactions on one or more nucleic acid molecules in a sample. The isolation or purification reactions may include contacting a sample with one or more beads or bead sets. The isolation or purification reaction may include one or more hybridization reactions, enrichment reactions, amplification reactions, sequencing reactions, or a combination thereof. The isolation or purification reaction may include the use of one or more separators. The one or more separators may include a magnetic separator. The isolation or purification reaction may include separating bead bound nucleic acid molecules from bead free nucleic acid molecules. The isolation or purification reaction may include separating capture probe hybridized nucleic acid molecules from capture probe free nucleic acid molecules. The isolation or purification reaction may include separating a first subset of nucleic acid molecules from a second subset of nucleic acid molecules, wherein the first subset of nucleic acid molecules differ from the second subset on nucleic acid molecules by mean size, mean GC content, genomic regions, or a combination thereof.

Some embodiments may include conducting one or more elution reactions on one or more nucleic acid molecules in a sample. The elution reactions may include contacting a sample with one or more beads or bead sets. The elution reaction may include separating bead bound nucleic acid molecules from bead free nucleic acid molecules. The elution reaction may include separating capture probe hybridized nucleic acid molecules from capture probe free nucleic acid molecules. The elution reaction may include separating a first subset of nucleic acid molecules from a second subset of nucleic acid molecules, wherein the first subset of nucleic acid molecules differ from the second subset on nucleic acid molecules by mean size, mean GC content, genomic regions, or a combination thereof.

Some embodiments may include one or more fragmentation reactions. The fragmentation reactions may include fragmenting one or more nucleic acid molecules in a sample or subset of nucleic acid molecules to produce one or more fragmented nucleic acid molecules. The one or more nucleic acid molecules may be fragmented by sonication, needle shear, nebulisation, shearing (e.g., acoustic shearing, mechanical shearing, point-sink shearing), passage through a French pressure cell, or enzymatic digestion. Enzymatic digestion may occur by nuclease digestion (e.g., micrococcal nuclease digestion, endonucleases, exonucleases, RNAse H or DNase I). Fragmentation of the one or more nucleic acid molecules may result in fragment sized of about 100 base pairs to about 2000 base pairs, about 200 base pairs to about 1500 base pairs, about 200 base pairs to about 1000 base pairs, about 200 base pairs to about 500 base pairs, about 500 base pairs to about 1500 base pairs, and about 500 base pairs to about 1000 base pairs. The one or more fragmentation reactions may result in fragment sized of about 50 base pairs to about 1000 base pairs. The one or more fragmentation reactions may result in fragment sized of about 100 base pairs, 150 base pairs, 200 base pairs, 250 base pairs, 300 base pairs, 350 base pairs, 400 base pairs, 450 base pairs, 500 base pairs, 550 base pairs, 600 base pairs, 650 base pairs, 700 base pairs, 750 base pairs, 800 base pairs, 850 base pairs, 900 base pairs, 950 base pairs, 1000 base pairs or more.

Fragmenting the one or more nucleic acid molecules may include mechanical shearing of the one or more nucleic acid molecules in the sample for a period of time. The fragmentation reaction may occur for at least about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more seconds.

Fragmenting the one or more nucleic acid molecules may include contacting a nucleic acid sample with one or more beads. Fragmenting the one or more nucleic acid molecules may include contacting the nucleic acid sample with a plurality of beads, wherein the ratio of the volume of the plurality of beads to the volume of nucleic acid sample is about 0.10, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00 or more. Fragmenting the one or more nucleic acid molecules may include contacting the nucleic acid sample with a plurality of beads, wherein the ratio of the volume of the plurality of beads to the volume of nucleic acid is about 2.00, 1.90, 1.80, 1.70, 1.60, 1.50, 1.40, 1.30, 1.20, 1.10, 1.00, 0.90, 0.80, 0.70, 0.60, 0.50, 0.40, 0.30, 0.20, 0.10, 0.05, 0.04, 0.03, 0.02, 0.01 or less.

Some embodiments may include conducting one or more detection reactions on one or more nucleic acid molecules in a sample. Detection reactions may include one or more sequencing reactions. Alternatively, conducting a detection reaction includes optical sensing, electrical sensing, or a combination thereof. Optical sensing may include optical sensing of a photoilluminscence photon emission, fluorescence photon emission, pyrophosphate photon emission, chemiluminescence photon emission, or a combination thereof. Electrical sensing may include electrical sensing of an ion concentration, ion current modulation, nucleotide electrical field, nucleotide tunneling current, or a combination thereof.

Some embodiments may include conducting one or more quantification reactions on one or more nucleic acid molecules in a sample. Quantification reactions may include sequencing, PCR, qPCR, digital PCR, or a combination thereof.

Some embodiments may include one or more samples. Some embodiments may include 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 or more samples. The sample may be derived from a subject. The two or more samples may be derived from a single subject. The two or more samples may be derived from t2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 or more different subjects. The subject may be a mammal, reptiles, amphibians, avians, and fish. The mammal may be a human, ape, orangutan, monkey, chimpanzee, cow, pig, horse, rodent, bird, reptile, dog, cat, or other animal. A reptile may be a lizard, snake, alligator, turtle, crocodile, and tortoise. An amphibian may be a toad, frog, newt, and salamander. Examples of avians include, but are not limited to, ducks, geese, penguins, ostriches, and owls. Examples of fish include, but are not limited to, catfish, eels, sharks, and swordfish. Preferably, the subject is a human. The subject may suffer from a disease or condition (e.g., a cancer).

The two or more samples may be collected over 1, 2, 3, 4, 5, 6, 7, 8, 9, 10, 11, 12, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

The sample may be from a body fluid, cell, skin, tissue, organ, or combination thereof. The sample may be a blood, plasma, a blood fraction, saliva, sputum, urine, semen, transvaginal fluid, cerebrospinal fluid, stool, a cell or a tissue biopsy. The sample may be from an adrenal gland, appendix, bladder, brain, ear, esophagus, eye, gall bladder, heart, kidney, large intestine, liver, lung, mouth, muscle, nose, pancreas, parathyroid gland, pineal gland, pituitary gland, skin, small intestine, spleen, stomach, thymus, thyroid gland, trachea, uterus, vermiform appendix, cornea, skin, heart valve, artery, or vein.

The samples may include one or more nucleic acid molecules. The nucleic acid molecule may be a DNA molecule, RNA molecule (e.g. mRNA, cRNA or miRNA), and DNA/RNA hybrids.

Examples of DNA molecules include, but are not limited to, double-stranded DNA, single-stranded DNA, single-stranded DNA hairpins, cDNA, genomic DNA. The nucleic acid may be an RNA molecule, such as a double-stranded RNA, single-stranded RNA, ncRNA, RNA hairpin, and mRNA. Examples of ncRNA include, but are not limited to, siRNA, miRNA, snoRNA, piRNA, tiRNA, PASR, TASR, aTASR, TSSa-RNA, snRNA, RE-RNA, uaRNA, x-ncRNA, hY RNA, usRNA, snaR, and vtRNA.

Some embodiments may include one or more containers. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more containers. The one or more containers may be different, similar, identical, or a combination thereof. Examples of containers include, but are not limited to, plates, microplates, PCR plates, wells, microwells, tubes, Eppendorf tubes, vials, arrays, microarrays, and chips.

Some embodiments may include one or more reagents. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more reagents. The one or more reagents may be different, similar, identical, or a combination thereof. The reagents may improve the efficiency of the one or more assays. Reagents may improve the stability of the nucleic acid molecule or variant or derivative thereof. Reagents may include, but are not limited to, enzymes, proteases, nucleases, molecules, polymerases, reverse transcriptases, ligases, and chemical compounds. Some embodiments may include conducting an assay including one or more antioxidants. Generally, antioxidants are molecules that inhibit oxidation of another molecule. Examples of antioxidants include, but are not limited to, ascorbic acid (e.g., vitamin C), glutathione, lipoic acid, uric acid, carotenes, a-tocopherol (e.g., vitamin E), ubiquinol (e.g., coenzyme Q), and vitamin A.

Some embodiments may include one or more buffers or solutions. The one or more buffers or solutions may be different, similar, identical, or a combination thereof. The buffers or solutions may improve the efficiency of the one or more assays. Buffers or solutions may improve the stability of the nucleic acid molecule or variant or derivative thereof. Buffers or solutions may include, but are not limited to, wash buffers, elution buffers, and hybridization buffers.

Some embodiments may include one or more beads, a plurality of beads, or one or more bead sets. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more beads or bead sets. The one or more beads or bead sets may be different, similar, identical, or a combination thereof. The beads may be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads. In some aspects, the one or more beads include one or more Ampure beads. Alternatively, or additionally, the one or more beads include AMPure XP beads.

Some embodiments may include one or more primers, a plurality of primers, or one or more primer sets. The primers may further include one or more linkers. The primers may further include or more labels. The primers may be used in one or more assays. For example, the primers are used in one or more sequencing reactions, amplification reactions, or a combination thereof. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more primers or primer sets. The primers may include about 100 nucleotides. The primers may include between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects, the primers include between about 80 nucleotides to about 100 nucleotides. The one or more primers or primer sets may be different, similar, identical, or a combination thereof.

The primers may hybridize to at least a portion of the one or more nucleic acid molecules or variant or derivative thereof in the sample or subset of nucleic acid molecules. The primers may hybridize to one or more genomic regions. The primers may hybridize to different, similar, and/or identical genomic regions. The one or more primers may be at least about 50%, 55%, 60%, 65%, 70%, 75%, 80%, 85%, 90%, 95%, 97%, 99% or more complementary to the one or more nucleic acid molecules or variant or derivative thereof.

The primers may include one or more nucleotides. The primers may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more nucleotides. The primers may include about 100 nucleotides. The primers may include between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects, the primers include between about 80 nucleotides to about 100 nucleotides.

The plurality of primers or the primer sets may include two or more primers with identical, similar, and/or different sequences, linkers, and/or labels. For example, two or more primers include identical sequences. In another example, two or more primers include similar sequences. In yet another example, two or more primers include different sequences. The two or more primers may further include one or more linkers. The two or more primers may further include different linkers. The two or more primers may further include similar linkers. The two or more primers may further include identical linkers. The two or more primers may further include one or more labels. The two or more primers may further include different labels. The two or more primers may further include similar labels. The two or more primers may further include identical labels.

The capture probes, primers, labels, and/or beads may include one or more nucleotides. The one or more nucleotides may include RNA, DNA, a mix of DNA and RNA residues or their modified analogs such as 2′-OMe, or 2′-fluoro (2′-F), locked nucleic acid (LNA), or abasic sites.

Some embodiments may include one or more labels. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more labels. The one or more labels may be different, similar, identical, or a combination thereof.

Examples of labels include, but are not limited to, chemical, biochemical, biological, colorimetric, enzymatic, fluorescent, and luminescent labels, which are well known in the art. The label include a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof.

The label may be a chemical label. Examples of chemical labels can include, but are not limited to, biotin and radiosiotypes (e.g., iodine, carbon, phosphate, hydrogen).

The methods, kits, and compositions disclosed herein may include a biological label. The biological labels may include metabolic labels, including, but not limited to, bioorthogonal azide-modified amino acids, sugars, and other compounds.

The methods, kits, and compositions disclosed herein may include an enzymatic label. Enzymatic labels can include, but are not limited to, horseradish peroxidase (HRP), alkaline phosphatase (AP), glucose oxidase, and 0-galactosidase. The enzymatic label may be luciferase.

The methods, kits, and compositions disclosed herein may include a fluorescent label. The fluorescent label may be an organic dye (e.g., FITC), biological fluorophore (e.g., green fluorescent protein), or quantum dot. A non-limiting list of fluorescent labels includes fluorescein isothiocyante (FITC), DyLight Fluors, fluorescein, rhodamine (tetramethyl rhodamine isothiocyanate, TRITC), coumarin, Lucifer Yellow, and BODIPY. The label may be a fluorophore. Exemplary fluorophores include, but are not limited to, indocarbocyanine (C3), indodicarbocyanine (C5), Cy3, Cy3.5, Cy5, Cy5.5, Cy7, Texas Red, Pacific Blue, Oregon Green 488, Alexa Fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX™), LIZ™, VIC™ NED™ PET™, SYBR, PicoGreen, RiboGreen, and the like. The fluorescent label may be a green fluorescent protein (GFP), red fluorescent protein (RFP), yellow fluorescent protein, phycobiliproteins (e.g., allophycocyanin, phycocyanin, phycoerythrin, and phycoerythrocyanin).

Some embodiments may include one or more linkers. Some embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more linkers. The one or more linkers may be different, similar, identical, or a combination thereof.

Suitable linkers include any chemical or biological compound capable of attaching to a label, primer, and/or capture probe disclosed herein. If the linker attaches to both the label and the primer or capture probe, then a suitable linker would be capable of sufficiently separating the label and the primer or capture probe. Suitable linkers would not significantly interfere with the ability of the primer and/or capture probe to hybridize to a nucleic acid molecule, portion thereof, or variant or derivative thereof. Suitable linkers would not significantly interfere with the ability of the label to be detected. The linker may be rigid. The linker may be flexible. The linker may be semi rigid. The linker may be proteolytically stable (e.g., resistant to proteolytic cleavage). The linker may be proteolytically unstable (e.g., sensitive to proteolytic cleavage). The linker may be helical. The linker may be non-helical. The linker may be coiled. The linker may be β-stranded. The linker may include a turn conformation. The linker may be a single chain. The linker may be a long chain. The linker may be a short chain. The linker may include at least about 5 residues, at least about 10 residues, at least about 15 residues, at least about 20 residues, at least about 25 residues, at least about 30 residues, or at least about 40 residues or more.

Examples of linkers include, but are not limited to, hydrazone, disulfide, thioether, and peptide linkers. The linker may be a peptide linker. The peptide linker may include a proline residue. The peptide linker may include an arginine, phenylalenine, threonine, glutamine, glutamate, or any combination thereof. The linker may be a heterobifunctional crosslinker.

Some embodiments may include conducting 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, or 50 or more assays on a sample including one or more nucleic acid molecules. The two or more assays may be different, similar, identical, or a combination thereof. For example, some embodiments include conducting two or more sequencing reactions. In another example, some embodiments include conducting two or more assays, wherein at least one of the two or more assays includes a sequencing reaction. In yet another example, some embodiments include conducting two or more assays, wherein at least two of the two or more assays includes a sequencing reaction and a hybridization reaction. The two or more assays may be performed sequentially, simultaneously, or a combination thereof. For example, the two or more sequencing reactions may be performed simultaneously. In another example, some embodiments include conducting a hybridization reaction, followed by a sequencing reaction. In yet another example, some embodiments include conducting two or more hybridization reactions simultaneously, followed by conducting two or more sequencing reactions simultaneously. The two or more assays may be performed by one or more devices. For example, two or more amplification reactions may be performed by a PCR machine. In another example, two or more sequencing reactions may be performed by two or more sequencers.

C. Devices

Some embodiments may include one or more devices. Some embodiments may include one or more assays including one or more devices. Some embodiments may include the use of one or more devices to perform one or more steps or assays. Some embodiments may include the use of one or more devices in one or more steps or assays. For example, conducting a sequencing reaction may include one or more sequencers. In another example, producing a subset of nucleic acid molecules may include the use of one or more magnetic separators. In yet another example, one or more processors may be used in the analysis of one or more nucleic acid samples. Exemplary devices include, but are not limited to, sequencers, thermocyclers, real-time PCR instruments, magnetic separators, transmission devices, hybridization chambers, electrophoresis apparatus, centrifuges, microscopes, imagers, fluorometers, luminometers, plate readers, computers, processors, and bioanalyzers.

Some embodiments may include one or more sequencers. The one or more sequencers may include one or more HiSeq, MiSeq, HiScan, Genome Analyzer IIx, SOLiD Sequencer, Ion Torrent PGM, 454 GS Junior, Pac Bio RS, or a combination thereof. The one or more sequencers may include one or more sequencing platforms. The one or more sequencing platforms may include GS FLX by 454 Life Technologies/Roche, Genome Analyzer by Solexa/Illumina, SOLiD by Applied Biosystems, CGA Platform by Complete Genomics, PacBio RS by Pacific Biosciences, or a combination thereof.

Some embodiments may include one or more thermocyclers. The one or more thermocyclers may be used to amplify one or more nucleic acid molecules. Some embodiments may include one or more real-time PCR instruments. The one or more real-time PCR instruments may include a thermal cycler and a fluorimeter. The one or more thermocyclers may be used to amplify and detect one or more nucleic acid molecules.

Some embodiments may include one or more magnetic separators. The one or more magnetic separators may be used for separation of paramagnetic and ferromagnetic particles from a suspension. The one or more magnetic separators may include one or more LifeStep™ biomagnetic separators, SPHERO™ FlexiMag separator, SPHERO™ MicroMag separator, SPHERO™ HandiMag separator, SPHERO™ MiniTube Mag separator, SPHERO™ UltraMag separator, DynaMag™ magnet, DynaMag™-2 Magnet, or a combination thereof.

Some embodiments may include one or more bioanalyzers. Generaly, a bioanalyzer is a chip-based capillary electrophoresis machine that can analyse RNA, DNA, and proteins. The one or more bioanalyzers may include Agilent's 2100 Bioanalyzer.

Some embodiments may include one or more processors. The one or more processors may analyze, compile, store, sort, combine, assess or otherwise process one or more data and/or results from one or more assays, one or more data and/or results based on or derived from one or more assays, one or more outputs from one or more assays, one or more outputs based on or derived from one or more assays, one or more outputs from one or data and/or results, one or more outputs based on or derived from one or more data and/or results, or a combination thereof. The one or more processors may transmit the one or more data, results, or outputs from one or more assays, one or more data, results, or outputs based on or derived from one or more assays, one or more outputs from one or more data or results, one or more outputs based on or derived from one or more data or results, or a combination thereof. The one or more processors may receive and/or store requests from a user. The one or more processors may produce or generate one or more data, results, outputs. The one or more processors may produce or generate one or more biomedical reports. The one or more processors may transmit one or more biomedical reports. The one or more processors may analyze, compile, store, sort, combine, assess or otherwise process information from one or more databases, one or more data or results, one or more outputs, or a combination thereof. The one or more processors may analyze, compile, store, sort, combine, assess or otherwise process information from 1, 2, 3, 4, 5, 6, 7, 8, 9, 10, 11, 12, 13, 14, 15, 16, 17, 18, 19, 20, 30 or more databases. The one or more processors may transmit one or more requests, data, results, outputs and/or information to one or more users, processors, computers, computer systems, memory locations, devices, databases, or a combination thereof. The one or more processors may receive one or more requests, data, results, outputs and/or information from one or more users, processors, computers, computer systems, memory locations, devices, databases or a combination thereof. The one or more processors may retrieve one or more requests, data, results, outputs and/or information from one or more users, processors, computers, computer systems, memory locations, devices, databases or a combination thereof.

Some embodiments may include one or more memory locations. The one or more memory locations may store information, data, results, outputs, requests, or a combination thereof. The one or more memory locations may receive information, data, results, outputs, requests, or a combination thereof from one or more users, processors, computers, computer systems, devices, or a combination thereof.

Methods described herein can be implemented with the aid of one or more computers and/or computer systems. A computer or computer system may include electronic storage locations (e.g., databases, memory) with machine-executable code for implementing the methods provided herein, and one or more processors for executing the machine-executable code.

The code can be pre-compiled and configured for use with a machine have a processer adapted to execute the code or can be compiled during runtime. The code can be supplied in a programming language that can be selected to enable the code to execute in a pre-compiled or as-compiled fashion.

The one or more computers and/or computer systems may analyze, compile, store, sort, combine, assess or otherwise process one or more data and/or results from one or more assays, one or more data and/or results based on or derived from one or more assays, one or more outputs from one or more assays, one or more outputs based on or derived from one or more assays, one or more outputs from one or data and/or results, one or more outputs based on or derived from one or more data and/or results, or a combination thereof. The one or more computers and/or computer systems may transmit the one or more data, results, or outputs from one or more assays, one or more data, results, or outputs based on or derived from one or more assays, one or more outputs from one or more data or results, one or more outputs based on or derived from one or more data or results, or a combination thereof. The one or more computers and/or computer systems may receive and/or store requests from a user. The one or more computers and/or computer systems may produce or generate one or more data, results, outputs. The one or more computers and/or computer systems may produce or generate one or more biomedical reports. The one or more computers and/or computer systems may transmit one or more biomedical reports. The one or more computers and/or computer systems may analyze, compile, store, sort, combine, assess or otherwise process information from one or more databases, one or more data or results, one or more outputs, or a combination thereof. The one or more computers and/or computer systems may analyze, compile, store, sort, combine, assess or otherwise process information from 1, 2, 3, 4, 5, 6, 7, 8, 9, 10, 11, 12, 13, 14, 15, 16, 17, 18, 19, 20, 30 or more databases. The one or more computers and/or computer systems may transmit one or more requests, data, results, outputs, and/or information to one or more users, processors, computers, computer systems, memory locations, devices, or a combination thereof. The one or more computers and/or computer systems may receive one or more requests, data, results, outputs, and/or information from one or more users, processors, computers, computer systems, memory locations, devices, or a combination thereof. The one or more computers and/or computer systems may retrieve one or more requests, data, results, outputs and/or information from one or more users, processors, computers, computer systems, memory locations, devices, databases or a combination thereof.

D. Databases

Some embodiments may include one or more databases. Some embodiments may include at least about 1, 2, 3, 4, 5, 6, 7, 8, 9, 10, 11, 12, 13, 14, 15, 16, 17, 18, 19, 20, 30 or more databases. The databases may include genomic, proteomic, pharmacogenomic, biomedical, and scientific databases. The databases may be publicly available databases. Alternatively, or additionally, the databases may include proprietary databases. The databases may be commercially available databases. The databases include, but are not limited to, Cosmic, GnomAD, Dbsnp, Mills Indels, MendelDB, PharmGKB, Varimed, Regulome, curated BreakSeq junctions, Online Mendelian Inheritance in Man (OMIM), Human Genome Mutation Database (HGMD), NCBI db SNP, NCBI RefSeq, GENCODE, GO (gene ontology), and Kyoto Encyclopedia of Genes and Genomes (KEGG).

Some embodiments may include analyzing one or more databases. Some embodiments may include analyzing at least about 1, 2, 3, 4, 5, 6, 7, 8, 9, 10, 11, 12, 13, 14, 15, 16, 17, 18, 19, 20, 30 or more databases. Analyzing the one or more databases may include one or more algorithms, computers, processors, memory locations, devices, or a combination thereof.

Some embodiments may include identifying one or more nucleic acid regions based on data and/or information from one or more databases. Some embodiments may include identifying one or more sets of nucleic acid regions based on data and/or information from one or more databases. Some embodiments may include identifying one or more nucleic acid regions and/or sets of nucleic acid regions based on data and/or information from at least about 2 or more databases. Some embodiments may include identifying one or more nucleic acid regions and/or sets of nucleic acid regions based on data and/or information from at least about 3 or more databases. Some embodiments may include identifying one or more nucleic acid regions and/or sets of nucleic acid regions based on data and/or information from at least about 4, 5, 6, 7, 8, 9, 10, 11, 12, 13, 14, 15, 16, 17, 18, 19, 20, 30 or more databases.

Some embodiments may include analyzing one or more results based on data and/or information from one or more databases. Some embodiments may include analyzing one or more sets of results based on data and/or information from one or more databases. Some embodiments may include analyzing one or more combined results based on data and/or information from one or more databases. Some embodiments may include analyzing one or more results, sets of results, and/or combined results based on data and/or information from at least about 2 or more databases. Some embodiments may include analyzing one or more results, sets of results, and/or combined results based on data and/or information from at least about 3 or more databases. Some embodiments may include analyzing one or more results, sets of results, and/or combined results based on data and/or information from at least about 4, 5, 6, 7, 8, 9, 10, 11, 12, 13, 14, 15, 16, 17, 18, 19, 20, 30 or more databases.

Some embodiments may include comparing one or more results based on data and/or information from one or more databases. Some embodiments may include comparing one or more sets of results based on data and/or information from one or more databases. Some embodiments may include comparing one or more combined results based on data and/or information from one or more databases. Some embodiments may include comparing one or more results, sets of results, and/or combined results based on data and/or information from at least about 2 or more databases. Some embodiments may include comparing one or more results, sets of results, and/or combined results based on data and/or information from at least about 3 or more databases. Some embodiments may include comparing one or more results, sets of results, and/or combined results based on data and/or information from at least about 4, 5, 6, 7, 8, 9, 10, 11, 12, 13, 14, 15, 16, 17, 18, 19, 20, 30 or more databases.

Some embodiments may include biomedical databases, genomic databases, biomedical reports, disease reports, case-control analysis, and rare variant discovery analysis based on data and/or information from one or more databases, one or more assays, one or more data or results, one or more outputs based on or derived from one or more assays, one or more outputs based on or derived from one or more data or results, or a combination thereof.

E. Analysis

Some embodiments may include one or more data, one or more data sets, one or more combined data, one or more combined data sets, one or more results, one or more sets of results, one or more combined results, or a combination thereof. The data and/or results may be based on or derived from one or more assays, one or more databases, or a combination thereof. Some embodiments may include analysis of the one or more data, one or more data sets, one or more combined data, one or more combined data sets, one or more results, one or more sets of results, one or more combined results, or a combination thereof. Some embodiments may include processing of the one or more data, one or more data sets, one or more combined data, one or more combined data sets, one or more results, one or more sets of results, one or more combined results, or a combination thereof.

Some embodiments may include at least one analysis and at least one processing of the one or more data, one or more data sets, one or more combined data, one or more combined data sets, one or more results, one or more sets of results, one or more combined results, or a combination thereof. Some embodiments may include one or more analyses and one or more processing of the one or more data, one or more data sets, one or more combined data, one or more combined data sets, one or more results, one or more sets of results, one or more combined results, or a combination thereof. Some embodiments may include at least 1, 2, 3, 4, 5, 6, 7, 8, 9, 10, 15, 20, 30, 40, 50, 60, 70, 80, 90, 100, 200, 300, 400, 500, 600, 700, 800, 900, 1000 or more distinct analyses of the one or more data, one or more data sets, one or more combined data, one or more combined data sets, one or more results, one or more sets of results, one or more combined results, or a combination thereof. Some embodiments may include at least 1, 2, 3, 4, 5, 6, 7, 8, 9, 10, 15, 20, 30, 40, 50, 60, 70, 80, 90, 100, 200, 300, 400, 500, 600, 700, 800, 900, 1000 or more distinct processing of the one or more data, one or more data sets, one or more combined data, one or more combined data sets, one or more results, one or more sets of results, one or more combined results, or a combination thereof. The one or more analyses and/or one or more processing may occur simultaneously, sequentially, or a combination thereof.

The one or more analyses and/or one or more processing may occur over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

Some embodiments may include one or more data. The one or more data may include one or more raw data based on or derived from one or more assays. The one or more data may include one or more raw data based on or derived from one or more databases. The one or more data may include at least partially analyzed data based on or derived from one or more raw data. The one or more data may include at least partially processed data based on or derived from one or more raw data. The one or more data may include fully analyzed data based on or derived from one or more raw data. The one or more data may include fully processed data based on or derived from one or more raw data. The data may include sequencing read data or expression data. The data may include biomedical, scientific, pharmacological, and/or genetic information.

Some embodiments may include one or more combined data. The one or more combined data may include two or more data. The one or more combined data may include two or more data sets. The one or more combined data may include one or more raw data based on or derived from one or more assays. The one or more combined data may include one or more raw data based on or derived from one or more databases. The one or more combined data may include at least partially analyzed data based on or derived from one or more raw data. The one or more combined data may include at least partially processed data based on or derived from one or more raw data. The one or more combined data may include fully analyzed data based on or derived from one or more raw data. The one or more combined data may include fully processed data based on or derived from one or more raw data. One or more combined data may include sequencing read data or expression data. One or more combined data may include biomedical, scientific, pharmacological, and/or genetic information.

Some embodiments may include one or more data sets. The one or more data sets may include one or more data. The one or more data sets may include one or more combined data. The one or more data sets may include one or more raw data based on or derived from one or more assays. The one or more data sets may include one or more raw data based on or derived from one or more databases. The one or more data sets may include at least partially analyzed data based on or derived from one or more raw data. The one or more data sets may include at least partially processed data based on or derived from one or more raw data. The one or more data sets may include fully analyzed data based on or derived from one or more raw data. The one or more data sets may include fully processed data based on or derived from one or more raw data. The data sets may include sequencing read data or expression data. The data sets may include biomedical, scientific, pharmacological, and/or genetic information.

Some embodiments may include one or more combined data sets. The one or more combined data sets may include two or more data. The one or more combined data sets may include two or more combined data. The one or more combined data sets may include two or more data sets. The one or more combined data sets may include one or more raw data based on or derived from one or more assays. The one or more combined data sets may include one or more raw data based on or derived from one or more databases. The one or more combined data sets may include at least partially analyzed data based on or derived from one or more raw data. The one or more combined data sets may include at least partially processed data based on or derived from one or more raw data. The one or more combined data sets may include fully analyzed data based on or derived from one or more raw data. The one or more combined data sets may include fully processed data based on or derived from one or more raw data. Some embodiments may further include further processing and/or analysis of the combined data sets. One or more combined data sets may include sequencing read data or expression data. One or more combined data sets may include biomedical, scientific, pharmacological, and/or genetic information.

Some embodiments may include one or more results. The one or more results may include one or more data, data sets, combined data, and/or combined data sets. The one or more results may be based on or derived from one or more data, data sets, combined data, and/or combined data sets. The one or more results may be produced from one or more assays. The one or more results may be based on or derived from one or more assays. The one or more results may be based on or derived from one or more databases. The one or more results may include at least partially analyzed results based on or derived from one or more data, data sets, combined data, and/or combined data sets. The one or more results may include at least partially processed results based on or derived from one or more data, data sets, combined data, and/or combined data sets. The one or more results may include at fully analyzed results based on or derived from one or more data, data sets, combined data, and/or combined data sets. The one or more results may include fully processed results based on or derived from one or more data, data sets, combined data, and/or combined data sets. The results may include sequencing read data or expression data. The results may include biomedical, scientific, pharmacological, and/or genetic information.

Some embodiments may include one or more sets of results. The one or more sets of results may include one or more data, data sets, combined data, and/or combined data sets. The one or more sets of results may be based on or derived from one or more data, data sets, combined data, and/or combined data sets. The one or more sets of results may be produced from one or more assays. The one or more sets of results may be based on or derived from one or more assays. The one or more sets of results may be based on or derived from one or more databases. The one or more sets of results may include at least partially analyzed sets of results based on or derived from one or more data, data sets, combined data, and/or combined data sets. The one or more sets of results may include at least partially processed sets of results based on or derived from one or more data, data sets, combined data, and/or combined data sets. The one or more sets of results may include at fully analyzed sets of results based on or derived from one or more data, data sets, combined data, and/or combined data sets. The one or more sets of results may include fully processed sets of results based on or derived from one or more data, data sets, combined data, and/or combined data sets. The sets of results may include sequencing read data or expression data. The sets of results may include biomedical, scientific, pharmacological, and/or genetic information.

Some embodiments may include one or more combined results. The combined results may include one or more results, sets of results, and/or combined sets of results. The combined results may be based on or derived from one or more results, sets of results, and/or combined sets of results. The one or more combined results may include one or more data, data sets, combined data, and/or combined data sets. The one or more combined results may be based on or derived from one or more data, data sets, combined data, and/or combined data sets. The one or more combined results may be produced from one or more assays. The one or more combined results may be based on or derived from one or more assays. The one or more combined results may be based on or derived from one or more databases. The one or more combined results may include at least partially analyzed combined results based on or derived from one or more data, data sets, combined data, and/or combined data sets. The one or more combined results may include at least partially processed combined results based on or derived from one or more data, data sets, combined data, and/or combined data sets. The one or more combined results may include at fully analyzed combined results based on or derived from one or more data, data sets, combined data, and/or combined data sets. The one or more combined results may include fully processed combined results based on or derived from one or more data, data sets, combined data, and/or combined data sets. The combined results may include sequencing read data or expression data. The combined results may include biomedical, scientific, pharmacological, and/or genetic information.

Some embodiments may include one or more combined sets of results. The combined sets of results may include one or more results, sets of results, and/or combined results. The combined sets of results may be based on or derived from one or more results, sets of results, and/or combined results. The one or more combined sets of results may include one or more data, data sets, combined data, and/or combined data sets. The one or more combined sets of results may be based on or derived from one or more data, data sets, combined data, and/or combined data sets. The one or more combined sets of results may be produced from one or more assays. The one or more combined sets of results may be based on or derived from one or more assays. The one or more combined sets of results may be based on or derived from one or more databases. The one or more combined sets of results may include at least partially analyzed combined sets of results based on or derived from one or more data, data sets, combined data, and/or combined data sets. The one or more combined sets of results may include at least partially processed combined sets of results based on or derived from one or more data, data sets, combined data, and/or combined data sets. The one or more combined sets of results may include at fully analyzed combined sets of results based on or derived from one or more data, data sets, combined data, and/or combined data sets. The one or more combined sets of results may include fully processed combined sets of results based on or derived from one or more data, data sets, combined data, and/or combined data sets. The combined sets of results may include sequencing read data or expression data. The combined sets of results may include biomedical, scientific, pharmacological, and/or genetic information.

Some embodiments may include one or more outputs, sets of outputs, combined outputs, and/or combined sets of outputs. The methods, libraries, kits and systems herein may include producing one or more outputs, sets of outputs, combined outputs, and/or combined sets of outputs. The sets of outputs may include one or more outputs, one or more combined outputs, or a combination thereof. The combined outputs may include one or more outputs, one or more sets of outputs, one or more combined sets of outputs, or a combination thereof. The combined sets of outputs may include one or more outputs, one or more sets of outputs, one or more combined outputs, or a combination thereof. The one or more outputs, sets of outputs, combined outputs, and/or combined sets of outputs may be based on or derived from one or more data, one or more data sets, one or more combined data, one or more combined data sets, one or more results, one or more sets of results, one or more combined results, or a combination thereof. The one or more outputs, sets of outputs, combined outputs, and/or combined sets of outputs may be based on or derived from one or more databases. The one or more outputs, sets of outputs, combined outputs, and/or combined sets of outputs may include one or more biomedical reports, biomedical outputs, rare variant outputs, pharmacogenetic outputs, population study outputs, case-control outputs, biomedical databases, genomic databases, disease databases, net content.

Some embodiments may include one or more biomedical outputs, one or more sets of biomedical outputs, one or more combined biomedical outputs, one or more combined sets of biomedical outputs. The methods, libraries, kits and systems herein may include producing one or more biomedical outputs, one or more sets of biomedical outputs, one or more combined biomedical outputs, one or more combined sets of biomedical outputs. The sets of biomedical outputs may include one or more biomedical outputs, one or more combined biomedical outputs, or a combination thereof. The combined biomedical outputs may include one or more biomedical outputs, one or more sets of biomedical outputs, one or more combined sets of biomedical outputs, or a combination thereof. The combined sets of biomedical outputs may include one or more biomedical outputs, one or more sets of biomedical outputs, one or more combined biomedical outputs, or a combination thereof. The one or more biomedical outputs, one or more sets of biomedical outputs, one or more combined biomedical outputs, one or more combined sets of biomedical outputs may be based on or derived from one or more data, one or more data sets, one or more combined data, one or more combined data sets, one or more results, one or more sets of results, one or more combined results, one or more outputs, one or more sets of outputs, one or more combined outputs, one or more sets of combined outputs, or a combination thereof. The one or more biomedical outputs may include biomedical information of a subject. The biomedical information of the subject may predict, diagnose, and/or prognose one or more biomedical features. The one or more biomedical features may include the status of a disease or condition, genetic risk of a disease or condition, reproductive risk, genetic risk to a fetus, risk of an adverse drug reaction, efficacy of a drug therapy, prediction of optimal drug dosage, transplant tolerance, or a combination thereof.

Some embodiments may include one or more biomedical reports. The methods, libraries, kits and systems herein may include producing one or more biomedical reports. The one or more biomedical reports may be based on or derived from one or more data, one or more data sets, one or more combined data, one or more combined data sets, one or more results, one or more sets of results, one or more combined results, one or more outputs, one or more sets of outputs, one or more combined outputs, one or more sets of combined outputs, one or more biomedical outputs, one or more sets of biomedical outputs, combined biomedical outputs, one or more sets of biomedical outputs, or a combination thereof. The biomedical report may predict, diagnose, and/or prognose one or more biomedical features. The one or more biomedical features may include the status of a disease or condition, genetic risk of a disease or condition, reproductive risk, genetic risk to a fetus, risk of an adverse drug reaction, efficacy of a drug therapy, prediction of optimal drug dosage, transplant tolerance, or a combination thereof.

Some embodiments may also include the transmission of one or more data, information, results, outputs, reports or a combination thereof. For example, data/information based on or derived from the one or more assays are transmitted to another device and/or instrument. In another example, the data, results, outputs, biomedical outputs, biomedical reports, or a combination thereof are transmitted to another device and/or instrument. The information obtained from an algorithm may also be transmitted to another device and/or instrument. Information based on the analysis of one or more databases may be transmitted to another device and/or instrument. Transmission of the data/information may include the transfer of data/information from a first source to a second source. The first and second sources may be in the same approximate location (e.g., within the same room, building, block, campus). Alternatively, first and second sources may be in multiple locations (e.g., multiple cities, states, countries, continents, etc). The data, results, outputs, biomedical outputs, biomedical reports can be transmitted to a patient and/or a healthcare provider.

Transmission may be based on the analysis of one or more data, results, information, databases, outputs, reports, or a combination thereof. For example, transmission of a second report is based on the analysis of a first report. Alternatively, transmission of a report is based on the analysis of one or more data or results. Transmission may be based on receiving one or more requests. For example, transmission of a report may be based on receiving a request from a user (e.g., patient, healthcare provider, individual).

Transmission of the data/information may include digital transmission or analog transmission. Digital transmission may include the physical transfer of data (a digital bit stream) over a point-to-point or point-to-multipoint communication channel. Examples of such channels are copper wires, optical fibres, wireless communication channels, and storage media. The data may be represented as an electromagnetic signal, such as an electrical voltage, radiowave, microwave, or infrared signal.

Analog transmission may include the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

Some embodiments may include one or more sample identifiers. The sample identifiers may include labels, barcodes, and other indicators which can be linked to one or more samples and/or subsets of nucleic acid molecules. Some embodiments may include one or more processors, one or more memory locations, one or more computers, one or more monitors, one or more computer software, one or more algorithms for linking data, results, outputs, biomedical outputs, and/or biomedical reports to a sample.

Some embodiments may include a processor for correlating the expression levels of one or more nucleic acid molecules with a prognosis of disease outcome. Some embodiments may include one or more of a variety of correlative techniques, including lookup tables, algorithms, multivariate models, and linear or nonlinear combinations of expression models or algorithms. The expression levels may be converted to one or more likelihood scores, reflecting a likelihood that the patient providing the sample may exhibit a particular disease outcome. The models and/or algorithms can be provided in machine readable format and can optionally further designate a treatment modality for a patient or class of patients.

In some cases, the methods and systems as described herein are used to generate an output including detection and/or quantitation of genomic DNA regions such as a region containing a DNA polymorphism (e.g., a germline variant or a somatic variant). In some cases, the detection of the one or more genomic regions is based on one or more algorithms, depending on the source of data inputs or databases that are described elsewhere in the instant specification. Each of the one or more algorithms can be used to receive, combine and generate data including detection of genomic regions (i.e., polymorphisms). In some embodiments, the instant method and system can include detection of the genomic regions that is based on one or more, two or more, three or more, four or more, five or more, six or more, seven or more, eight or more, nine or more or ten or more algorithms. The algorithms can be machine-learning algorithms, computer-implemented algorithms, machine-executed algorithms, automatic algorithms and the like.

The resulting data for each nucleic acid sample can be analyzed using feature selection techniques including filter techniques which assess the relevance of features by examining the intrinsic properties of the data, wrapper methods which embed the model hypothesis within a feature subset search, and embedded techniques in which the search for an optimal set of features is built into an algorithm or model.

In some cases, the detection of the one or more genomic regions is based on one or more statistical models. Statistical models or filtering techniques useful in the methods of the present invention include (1) parametric methods such as the use of two sample t-tests, ANOVA analyses, Bayesian frameworks, and Gamma distribution models, (2) model free methods such as the use of Wilcoxon rank sum tests, between-within class sum of squares tests, rank products methods, random permutation methods, or TNoM which involves setting a threshold point for fold-change differences in expression between two datasets and then detecting the threshold point in each gene that minimizes the number of missclassifications, and (3) multivariate methods such as bivariate methods, correlation based feature selection methods (CFS), minimum redundancy maximum relevance methods (MRMR), Markov blanket filter methods, Markov models, Hidden Markov Model (HMM), and uncorrelated shrunken centroid methods. In some cases, the Hidden Markov Model (HMM) is given an internal state, wherein the internal state is set according to an overall copy number of a chromosome in the first or second nucleic acid sample. In an instance, for a diploid chromosome, the HMI's internal states can be homozygous deletion (locally zero copies), heterozygous deletion (locally one copy), normal (locally two copies), duplication (more than two copies), and reference Gap (present as a state to distinguish gaps from Homozygous deletions). In another instance, for a Haploid chromosome (e.g., X or Y in a male), the HMM's internal states can be homozygous deletion (locally zero copies), normal (locally two copies), duplication (more than two copies), and reference Gap (present as a state to distinguish gaps from Homozygous deletions). For example, for a Haploid chromosome, there may be no heterozygous deletion state available. In another instance, for trisomic and/or tetrasomic, additional intermediate the HMM states may have an additional intermediate state, wherein the intermediate state can account for the various CNV possibilities. In another embodiment, the Hidden Markov Model is used to filter the output by examination of measured insert-sizes of reads near a detected feature's breakpoint(s).

Other models or algorithms useful in the methods of the present invention include sequential search methods, genetic algorithms, estimation of distribution algorithms, random forest algorithms, weight vector of support vector machine algorithms, weights of logistic regression algorithms, and the like. Bioinformatics. 2007 Oct. 1;23(19):2507-17 provides an overview of the relative merits of the algorithms or models provided above for the analysis of data. Illustrative algorithms include but are not limited to methods that reduce the number of variables such as principal component analysis algorithms, partial least squares methods, independent component analysis algorithms, methods that handle large numbers of variables directly such as statistical methods, and methods based on machine learning techniques. Statistical methods include penalized logistic regression, prediction analysis of microarrays (PAM), methods based on shrunken centroids, support vector machine analysis, and regularized linear discriminant analysis. Machine learning techniques include fully connected neural networks, convolutional neural networks, 1D convolutional neural networks, 2D convolutional neural networks, gradient boosting decision trees (e.g., XGBoost framework, LightGBM framework), bagging procedures, boosting procedures, random forest algorithms, and combinations thereof. Cancer Inform. 2008; 6: 77-97 provides an overview of the techniques provided above for the analysis of data. In some embodiments, the trained machine-learning model includes a gradient boosting decision tree (e.g., including a LightGBM framework). In some embodiments, the trained machine-learning model includes a convolutional neural network (e.g., a 1D convolutional neural network or a 2D convolutional neural network). In some embodiments, the trained machine-learning model includes a fully connected neural network.

Machine learning can include deep learning. Deep learning can be used to capture the internal structure of increasingly larger and high-dimensional data sets (e.g., data from nucleic acid sequencing). Deep models can enable the discovery of high-level features, improving performances over traditional models, increasing interpretability, and providing additional understanding about the structure of the biological data.

The trained machine-learning model can include a fully connected neural network. A fully connected neural network can include a series of fully connected layers. Each output dimension can depend on each input dimension. A fully connected neural network can be a feed-forward network.

The trained machine-learning model can include a convolutional neural network. A convolutional neural network can rely on local connections and tied weights across the units followed by feature pooling (subsampling) to obtain translation invariant descriptors. The basic convolutional neural network architecture can include one convolutional and pooling layer, optionally followed by a fully connected layer for supervised prediction. In practice, convolutional neural networks can be composed of multiple (e.g., >10) convolutional and pooling layers to better model the input space. In some cases, convolutional neural networks require a large data set to be well trained. In some cases, convolutional neural networks can use less parameters than a fully connected neural network by computing convolution on small regions of the input space and by sharing parameters between regions. A convolutional neural network can be a one dimensional (1D) convolutional neural network. A convolutional neural network can be a two dimensional (2D) convolutional neural network. In some embodiments, a convolutional neural network includes three or more dimensions.

The trained machine-learning model can include a gradient-boosted decision tree. Gradient boosting is a machine learning technique that can be used for regression and classification problems, which can produce a prediction model in the form of an ensemble of weak prediction models, e.g., decision trees. A gradient boosted decision tree can include, for example, an XGBoost framework or a LightGBM framework.

A machine-learning model can include hyperparameters. A hyperparameters can be a configuration that is external to the model and whose value cannot be estimated from data. Hyperparameters can be tuned, e.g., tuned for a given predictive modeling problem. In some cases, a hyperparameter is used in processes to help estimate model parameters. In some cases, a hyperparameter can be specified by a practitioner. In some cases, a hyperparameter can be set using heuristics.

In some embodiments, an HMM-based detection algorithm can “segmentally” detect a large or substantially large CNV. In some cases, due to fluctuations in the coverage signal, there may be small detection gaps along the length of the true CNV. In an example, a 1 megabasepairs (Mbp) deletion may be detected as a small number of separate nominal detections, with small gaps between them. To mitigate this, a merge operation can be employed that identifies pairs of adjacent detections which are separated by a gap that is smaller than either of the two bracketing detections. The merge operation then measures the median coverage level in the gap. If the median coverage passes a predefined threshold, then the two detections are merged into a single large detection that spans the two original detections (including the enclosed detection gap). In an example, the true feature spans both detections, and the gap is a statistical artifact. Using real sequencing data of samples that are known to have large CNVs, this merge operation can permit a substantially better fidelity with respect to the true properties of the CNVs.

Methods and systems provided herein may further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420).

In some embodiments of the present invention, a diagonal linear discriminant analysis, k-nearest neighbor algorithm, support vector machine (SVM) algorithm, linear support vector machine, random forest algorithm, or a probabilistic model-based method or a combination thereof is provided for the detection of one or more genomic regions. In some embodiments, identified markers that distinguish samples (e.g., diseased versus normal) or distinguish genomic regions (e.g., copy number variation versus. normal) are selected based on statistical significance of the difference in expression levels between classes of interest. In some cases, the statistical significance is adjusted by applying a Benjamini Hochberg or another correction for false discovery rate (FDR).

In some cases, the algorithm may be supplemented with a meta-analysis approach such as that described by Fishel and Kaufman et al. 2007 Bioinformatics 23(13): 1599-606. In some cases, the algorithm may be supplemented with a meta-analysis approach such as a repeatability analysis. In some cases, the repeatability analysis selects markers that appear in at least one predictive expression product marker set.

A statistical evaluation of the detection of the genomic regions may provide a quantitative value or values indicative of one or more of the following: the likelihood of diagnostic accuracy; the likelihood of disorder, disease, condition and the like; the likelihood of a particular disorder, disease or condition; and the likelihood of the success of a particular therapeutic intervention. Thus, a physician, who is not likely to be trained in genetics or molecular biology, need not understand the raw data. Rather, the data is presented directly to the physician in the form of the quantitative values to guide patient care. The results can be statistically evaluated using a number of methods known to the art including, but not limited to: the student's T test, the two-sided T test, Pearson rank sum analysis, Hidden Markov Model Analysis, analysis of q-q plots, principal component analysis, one way ANOVA, two way ANOVA, LIMMA, and the like.

F. Diseases or Conditions

Some embodiments may include predicting, diagnosing, and/or prognosing a status or outcome of a disease or condition in a subject based on one or more biomedical outputs. Predicting, diagnosing, and/or prognosing a status or outcome of a disease in a subject may include diagnosing a disease or condition, identifying a disease or condition, determining the stage of a disease or condition, assessing the risk of a disease or condition, assessing the risk of disease recurrence, assessing the efficacy of a drug, assessing risk of an adverse drug reaction, predicting optimal drug dosage, predicting drug resistance, or a combination thereof.

The samples disclosed herein may be from a subject suffering from a cancer. The sample may include malignant tissue, benign tissue, or a mixture thereof. The cancer may be a recurrent and/or refractory cancer. Examples of cancers include, but are not limited to, sarcomas, carcinomas, lymphomas or leukemias. In some cases, a sample including cancer tissue is obtained, but no matching normal sample is obtained. In some cases, no matching normal sample is available. In some cases, a matching normal sample is obtained (e.g., for training and testing of a model disclosed herein).

Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. The cancer may be a skin cancer, such as a basal cell carcinoma, squamous, melanoma, nonmelanoma, or actinic (solar) keratosis.

The cancer may be a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavitity (pleura) or lining of the abdomen (peritoneum). The mesothelioma may be due to asbestos exposure. The cancer may be a brain cancer, such as a glioblastoma.

The cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. The cancer may be a meningioma.

The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

Some embodiments may include treating and/or preventing a disease or condition in a subject based on one or more biomedical outputs. The one or more biomedical outputs may recommend one or more therapies. The one or more biomedical outputs may suggest, select, designate, recommend or otherwise determine a course of treatment and/or prevention of a disease or condition. The one or more biomedical outputs may recommend modifying or continuing one or more therapies. Modifying one or more therapies may include administering, initiating, reducing, increasing, and/or terminating one or more therapies. The one or more therapies include an anti-cancer, antiviral, antibacterial, antifungal, immunosuppressive therapy, or a combination thereof. The one or more therapies may treat, alleviate, or prevent one or more diseases or indications.

Examples of anti-cancer therapies include, but are not limited to, surgery, chemotherapy, radiation therapy, immunotherapy/biological therapy, photodynamic therapy. Anti-cancer therapies may include chemotherapeutics, monoclonal antibodies (e.g., rituximab, trastuzumab), cancer vaccines (e.g., therapeutic vaccines, prophylactic vaccines), gene therapy, or combination thereof.

G. Systems, Kits, and Libraries

Certain embodiments can be implemented by way of systems, kits, libraries, or a combination thereof. The methods of the invention may include one or more systems. Systems can be implemented by way of kits, libraries, or both. A system may include one or more components to perform any of the methods or any of the steps of Some embodiments. For example, a system may include one or more kits, devices, libraries, or a combination thereof. A system may include one or more sequencers, processors, memory locations, computers, computer systems, or a combination thereof. A system may include a transmission device.

A kit may include various reagents for implementing various operations disclosed herein, including sample processing and/or analysis operations. A kit may include instructions for implementing at least some of the operations disclosed herein. A kit may include one or more capture probes, one or more beads, one or more labels, one or more linkers, one or more devices, one or more reagents, one or more buffers, one or more samples, one or more databases, or a combination thereof.

A library may include one or more capture probes. A library may include one or more subsets of nucleic acid molecules. A library may include one or more databases. A library may be produced or generated from any of the methods, kits, or systems disclosed herein. A database library may be produced from one or more databases. A method for producing one or more libraries may include (a) aggregating information from one or more databases to produce an aggregated data set; (b) analyzing the aggregated data set; and (c) producing one or more database libraries from the aggregated data set.

It should be understood from the foregoing that, while particular implementations have been illustrated and described, various modifications may be made thereto and are contemplated herein. An embodiment of one aspect may be combined with or modified by an embodiment of another aspect. It is not intended that the invention(s) be limited by the specific examples provided within the specification. While the invention(s) has (or have) been described with reference to the aforementioned specification, the descriptions and illustrations of embodiments of the invention(s) herein are not meant to be construed in a limiting sense. Furthermore, it shall be understood that all aspects of the invention(s) are not limited to the specific depictions, configurations or relative proportions set forth herein which depend upon a variety of conditions and variables. Various modifications in form and detail of the embodiments of the invention(s) will be apparent to a person skilled in the art. It is therefore contemplated that the invention(s) shall also cover any such modifications, variations and equivalents.

### VI. Computing Environment

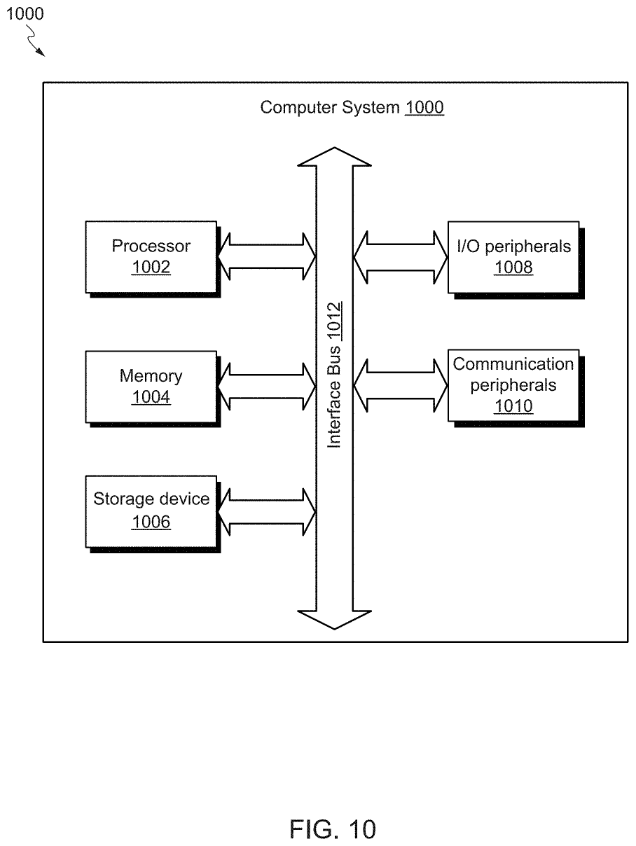

FIG. 10 illustrates an example of a computer system 1000 for implementing some of the embodiments disclosed herein. Computer system 1000 may have a distributed architecture, where some of the components (e.g., memory and processor) are part of an end user device and some other similar components (e.g., memory and processor) are part of a computer server. Computer system 1000 includes at least a processor 1002, a memory 1004, a storage device 1006, input/output (I/O) peripherals 1008, communication peripherals 1010, and an interface bus 1012. Interface bus 1012 is configured to communicate, transmit, and transfer data, controls, and commands among the various components of computer system 1000. Processor 1002 may include one or more processing units, such as CPUs, GPUs, TPUs, systolic arrays, or SIMD processors. Memory 1004 and storage device 1006 include computer-readable storage media, such as RAM, ROM, electrically erasable programmable read-only memory (EEPROM), hard drives, CD-ROMs, optical storage devices, magnetic storage devices, electronic non-volatile computer storage, for example, Flash® memory, and other tangible storage media. Any of such computer-readable storage media can be configured to store instructions or program codes embodying aspects. Memory 1004 and storage device 1006 also include computer-readable signal media. A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with computer system 1000.

Further, memory 1004 includes an operating system, programs, and applications. Processor 1002 is configured to execute the stored instructions and includes, for example, a logical processing unit, a microprocessor, a digital signal processor, and other processors. Memory 1004 and/or processor 1002 can be virtualized and can be hosted within another computing system of, for example, a cloud network or a data center. I/O peripherals 1008 include user interfaces, such as a keyboard, screen (e.g., a touch screen), microphone, speaker, other input/output devices, and computing components, such as graphical processing units, serial ports, parallel ports, universal serial buses, and other input/output peripherals. I/O peripherals 1008 are connected to processor 1002 through any of the ports coupled to interface bus 1012. Communication peripherals 1010 are configured to facilitate communication between computer system 1000 and other computing devices over a communications network and include, for example, a network interface controller, modem, wireless and wired interface cards, antenna, and other communication peripherals.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of Some embodiments may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “including,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A method comprising:
obtaining nucleic acid sequence data of a biological sample of a subject, wherein a reference biological sample of the subject that corresponds to the biological sample is unavailable, and wherein the reference biological sample includes non-tumor cells only;
aligning the nucleic acid sequence data to a reference genome;
identifying, based on the aligned nucleic acid sequence data of the biological sample, a set of candidate variants in said nucleic acid sequence data, wherein said set of candidate variants includes one or more somatic variants and one or more germline variants;
without using nucleic acid sequencing data of the reference biological sample of the subject, processing the set of candidate variants using a trained machine-learning model to identify the somatic variants; and
outputting a report that identifies the somatic variants.

2. The method of claim 1, wherein the biological sample is a tumor sample of the subject.

3. The method of claim 1, wherein the trained machine-learning model includes a gradient boosted decision tree.

4. The method of claim 1, wherein the trained machine-learning model includes two classification models.

5. The method of claim 1, wherein the trained machine-learning model includes a filtration model.

6. The method of claim 1, wherein the trained machine-learning model includes a rescue model.

7. The method of claim 1, wherein the trained machine-learning model is trained using training data corresponding to a set of matched tumor-normal pairs.

8. The method of claim 1, wherein the trained machine-learning model is trained by tuning one or more hyperparameters via a randomized search.

9. The method of claim 1, wherein the report identifies at least one biomarker.

10. The method of claim 1, wherein the report identifies at least one prognostic marker.

11. The method of claim 1, wherein the report identifies a presence or absence of the one or more somatic variants.

12. A system comprising:
one or more data processors; and
a non-transitory computer readable storage medium containing instructions which, when executed on the one or more data processors, cause the one or more data processors to perform one or more operations comprising:
obtaining nucleic acid sequence data of a biological sample of a subject, wherein a reference biological sample of the subject that corresponds to the biological sample is unavailable, and wherein the reference biological sample includes non-tumor cells only;
aligning the nucleic acid sequence data to a reference genome;
identifying, based on the aligned nucleic acid sequence data of the biological sample, a set of candidate variants in said nucleic acid sequence data, wherein said set of candidate variants includes one or more somatic variants and one or more germline variants;
without using nucleic acid sequencing data of the reference biological sample of the subject, processing the set of candidate variants using a trained machine-learning model to identify the somatic variants; and
outputting a report that identifies the somatic variants.

13. The system of claim 12, wherein the biological sample is a tumor sample of the subject.

14. The system of claim 12, wherein the trained machine-learning model includes one or more of a gradient boosted decision tree, a filtration model, or a rescue model.

15. The system of claim 12, wherein the trained machine-learning model is trained using training data corresponding to a set of matched tumor-normal pairs.

16. The system of claim 12, wherein the trained machine-learning model is trained by tuning one or more hyperparameters via a randomized search.

17. The system of claim 12, wherein the report identifies at least one biomarker.

18. The system of claim 12, wherein the report identifies at least one prognostic marker.

19. The system of claim 12, wherein the report identifies a presence or absence of the one or more somatic variants.

20. A computer-program product tangibly embodied in a non-transitory machine-readable storage medium, including instructions configured to cause one or more data processors to perform one or more operations comprising:
obtaining nucleic acid sequence data of a biological sample of a subject, wherein a reference biological sample of the subject that corresponds to the biological sample is unavailable, and wherein the reference biological sample includes non-tumor cells only;
aligning the nucleic acid sequence data to a reference genome;
identifying, based on the aligned nucleic acid sequence data of the biological sample, a set of candidate variants in said nucleic acid sequence data, wherein said set of candidate variants includes one or more somatic variants and one or more germline variants;
without using nucleic acid sequencing data of the reference biological sample of the subject, processing the set of candidate variants using a trained machine-learning model to identify the somatic variants; and
outputting a report that identifies the somatic variants.
